(19) United States
(12) Patent Application Publication
(10) Pub. No.: US 2024/0189560 A1
(43) Pub. Date: Jun. 13, 2024

(54) DEVICES, SYSTEMS, AND METHODS FOR DELIVERING TOPICAL MEDICATION

(71) Applicant: Daniel J. GOULD, Beverly Hills, CA (US)

(72) Inventor: Daniel J. GOULD, Beverly Hills, CA (US)

(21) Appl. No.: 18/499,983

(22) Filed: Nov. 1, 2023

Related U.S. Application Data
(63) Continuation of application No. 18/078,440, filed on Dec. 9, 2022.

(51) Int. Cl. A61M 35/00

(52) U.S. Cl. CPC A61M 35/003; A61M 2205/586

(57) ABSTRACT

Disclosed herein are devices, systems, and methods for applying and/or delivering topical compounds (e.g., topical medications) to the surface of mammalian tissue (e.g., skin, mucous membranes, and/or other tissue surfaces). Specifically, disclosed embodiments include a device that is configured to connect to one or more types of known syringes, and, in particular, to one or more types of known syringe tips. At least one embodiment of the device comprises a cap and a membrane, where the cap includes an open connector end for engaging and/or attaching to a syringe via a syringe tip, a delivery end opposite to the connector end, and a cap body disposed between, and connecting, the connector end and the delivery end. The delivery end can further be connected to the membrane by, for instance, inserting one end of the membrane into the delivery end.

### FIELD

The disclosure relates generally to devices, systems, and methods for delivering a topical compound, such as topical medications, to the surface of human tissue (e.g., skin). In particular, embodiments of the disclosure are directed to a device that is configured to connect (e.g., via a luer-style connector) to one or more existing types of syringe tips, thereby permitting a user to use the syringe to apply and/or deliver a topical medication for medical (e.g., to treat medical conditions) and/or cosmetic (e.g., to improve skin texture) purposes.

### BACKGROUND

Topical compounds are used in a variety of applications and industries to treat the surface of specific tissues, such as skin. For instance, in medicine, topical medications (e.g., processed human derived components, autogenous materials, allograft materials, steroids, anti-scarring medication, growth factors, and exosomes that encourage skin growth) are used to treat various medical conditions, such as, for instance, scarring, burns, and other skin conditions (e.g., hyperpigmentation, inflammation, fibrosis, and the like).

Additionally, topical compounds can be used for cosmetic benefits, such as, for instance, to prevent skin aging and to improve skin texture, quality, and/or clarity.

However, the application of topical compounds requires a device that is capable of both accurately and precisely applying such compounds. For instance, a medical professional or other user must be able to apply an accurate amount of a topical compound (e.g., a topical medication) to a precise area (e.g., a specific section of the skin surface). Such a device should also be easily controllable by the individual applying the compound in order to reduce waste, prevent overmedication, and the like.

Given the foregoing, there exists a significant need for devices, systems, and methods that can apply and/or deliver topical compounds, such as topical medications, to the surface of human tissue (e.g., skin). In particular, there is a need for devices that are compatible with known products (e.g., syringes) to provide improved control of the dispensation (e.g., the method and amount of dispensing) of such topical compounds.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In certain embodiments, the disclosed embodiments may include one or more of the features described herein.

Embodiments of the present disclosure are directed towards devices, systems, and methods for applying and/or delivering one or more topical compounds (e.g., topical medication) to the surface of mammalian tissue (e.g., skin, mucous membranes, and/or other tissue surfaces). At least one embodiment comprises a device that is configured to be engaged with and/or attached to a syringe, and specifically, a syringe tip.

In at least one embodiment, a device for applying and/or delivering one or more topical compounds is disclosed, the device comprising a cap and a membrane. The cap may be hollow and may comprise a first end (alternatively referred to herein as a “connector end”) for engaging with and/or attaching to a syringe (e.g., any known syringe) via the syringe tip, a second end (alternatively referred to herein as a “delivery end”) disposed opposite to the connector end, and a cap body disposed between, and connecting, the connector end and the delivery end. Additionally, the connector end and the delivery end may be fluidly connected. The aforementioned membrane may be connected, either detachably or fixably, to the delivery end.

In at least a further embodiment, the connector end comprises one or more connection mechanisms known for syringe tips, including, for instance, a luer-style connector, a slip-on connection, a push-on connection, an eccentric tip, a tapered tip, a catheter tip, and combinations thereof. The device may therefore, in at least one embodiment, be engaged with and/or attached to a tip for any known type of syringe, including, for instance, a disposable syringe, a safety syringe, an insulin syringe, a hypodermic syringe, a multi-shot needle syringe, a venom extraction syringe, an oral syringe, a dental syringe, a dose-sparing syringe, a glass syringe, a plastic syringe (e.g., a polyethylene syringe), and combinations thereof.

In at least an additional embodiment, a device for delivering one or more topical compounds to a surface of human tissue is disclosed. The device comprises a syringe, a cap, and a membrane. The syringe comprises a barrel and a syringe tip fluidly connected to the barrel. The cap comprises an open connector end, an open delivery end fluidly connected to the open connector end, and a cap body disposed between, and connecting, the open connector end and the open delivery end. The membrane comprises a delivery surface and is connected to the open delivery end such that the delivery surface closes the open delivery end.

In at least a further embodiment, the open connector end is releasably connected to the syringe tip such that one or more openings in the syringe tip are fluidly connected to the cap. Such releasable connection may be provided by a connector and/or connection mechanism such as, for instance, a luer-style connector, a slip-on connection, a push-on connection, an eccentric tip, a tapered tip, a catheter tip, and combinations thereof.

Thus, a user may use the device by, for instance, exerting force on a portion of the syringe (e.g., the barrel of the syringe) to move one or more topical compounds from the barrel of the syringe through the syringe tip and through the device, thereby extruding at least a portion of the one or more topical compounds through the attached membrane. Such portion may then be applied to the surface of human tissue. It should therefore be appreciated that, in at least one embodiment, the membrane is selectively permeable such that the membrane permits the one or more compounds inside the syringe to move through the membrane. Non-limiting examples of membrane materials include, for instance, one or more polymers (including synthetic/manmade polymers), one or more resins, etc. Thus, the membrane may be composed of, for instance, cellulose, gauze, and the like.

The one or more compounds may be one or more topical medications or other compounds, in any formulation or concentration, including, for instance, triamcinolone, fluorouracil (also known as “5-FU”), tranexamic acid (also known as “TXA”), azelaic acid, one or more bleaching agents (e.g., hydroquinone), one or more topical numbing agents (e.g., lidocaine, bupivacaine), one or more topical antibiotics, one or more dyes or stains (e.g., bonnet blue, gentian violet), one or more topical anti-inflammatory compounds such as nonsteroidal anti-inflammatory drugs (e.g., diclofenac, ibuprofen, Toradol), vitamins (e.g., vitamin A, vitamin B12, vitamin E, vitamin K), one or more topical steroids, one or more topical darkening agents for conditions such as hypopigmented scars (e.g., bimatoprost), anti-oxidants (e.g., N-acetyl-cysteine, glutathione), one or more micro-dosed compounds (e.g., botulinum toxin, hyaluronic acid, other compounds used in micro-needling reservoir treatments), one or more skincare compounds, one or more cosmetic compounds, one or more nutraceutical compounds, and combinations thereof.

Such compounds can be used to treat a variety of conditions, including, for instance, skin conditions such as scars, burns, hyperpigmentation, inflammation, and/or fibrosis. Purely as non-limiting examples, the device could be used to deliver (1) topical steroids to treat scars, (2) growth factors and anti-scarring medications to treat burns, (3) anti-aging compounds and/or vitamins to improve skin texture, quality, and/or clarity, (4) anti-inflammatory compounds to prevent and/or treat inflammation, and (5) medication or other treatment compounds as part of skin laser treatments, microneedling, dermal abrasion, and the like.

In at least an additional embodiment, a method is disclosed herein for delivering one or more topical compounds to a surface of human tissue. The method comprises obtaining an applicator having one or more topical compounds to be delivered, wherein the applicator comprises a cap and a membrane, wherein the cap comprises one end connected to the membrane, wherein the cap comprises an inner area; exerting force on the applicator to move the one or more topical compounds through the inner area and through the one end, such that at least one portion of the one or more topical compounds are extruded through the membrane; and positioning the applicator adjacent to a surface of human tissue such that the at least one portion of the one or more topical compounds contacts the surface, thereby delivering the one or more topical compounds.

In at least a further embodiment, the applicator comprises a syringe, which may be any syringe described herein and/or any known syringe. The syringe may comprise a barrel fluidly connected to a syringe tip, which may be any syringe tip described herein and/or any known syringe tip. The cap may further comprise a connector end that is configured to attach, either releasably or non-releasably, to the syringe tip, such that one or more openings in the syringe tip are fluidly connected to the connector end. Further, the cap may comprise a delivery end disposed opposite the connector end, wherein the delivery end is fluidly connected to the connector end.

In at least one embodiment, the method may further comprise engaging the membrane with the one end of the cap, and/or engaging the connector end of the cap with a syringe tip of a syringe.

In at least an additional embodiment, the aforementioned exerting force on the applicator may further comprise engaging the membrane with the one end of the cap, and/or engaging the connector end of the cap with a syringe tip of a syringe. The aforementioned exerting force on the applicator may additionally comprise (1) exerting the force on the barrel of the syringe, thereby moving the one or more topical compounds through the syringe tip and through the connector end, and/or (2) holding the applicator between a thumb and at least one finger of one hand.

Thus, a user of the method may, in at least one embodiment, hold the applicator in such a fashion to provide one or more benefits when compared with known methods of holding a syringe, in which the user's thumb contacts a top surface of the plunger of the syringe, rather than the syringe barrel, in order to exert force on the plunger to depress the plunger into the barrel, thereby ejecting the one or more compounds inside the barrel through a needle attached to the syringe tip.

Such aforementioned one or more benefits may include, for instance, more and/or better control of the amount of the one or more compounds applied, limiting the amount of the one or more compounds applied, more and/or better control of the surface area to which the one or more compounds are applied, prevention of overmedication, prevention of waste, and/or cost savings from avoiding excessive application amounts of the one or more compounds.

In at least a further embodiment, a device for applying and/or delivering topical compounds has different shapes, sizes, and/or form factors, including shapes, sizes, and/or form factors other than the cap and/or membrane described herein. Non-limiting examples of such devices include devices with a tapered end, an eccentric or eccentric-style end, an opening and/or a port (e.g., a side opening or port), an angled and/or beveled end, an end that narrows to an elongated and/or domed point, an applicator area that comprises one or more sponges and/or sponge-like surfaces, a plurality of microscopic (e.g., micron-sized and/or nanometer-sized) holes, a rounded end (e.g., similar to a ballpoint pen in shape and/or construction), a brush end (e.g., having one or more brushes and/or brush-like portions), a curved end shaped such that it is ergonomic and/or matches to one or more rounded surfaces on human tissues (e.g., rounded portions of the fingers, arms, etc.), a dropper and/or dropper-style end, an abrasive end (e.g., having one or more abrasive materials), a flat end that can be placed flat against a surface of human tissue (e.g., skin), an applicator end that further comprises an internal reservoir, an applicator end that has the same width as that of the syringe tip and/or the syringe and/or barrel attached to the syringe tip, an applicator end that further comprises one or more internal filters, and combinations thereof.

In at least one embodiment, any of the devices described herein may be lubricated along one or more internal surfaces and/or cavities.

In at least one embodiment, any of the devices described herein, including, without limitation, can have different lengths and/or thicknesses. Non-limiting examples of appropriate lengths include less than 1 centimeter (cm), 1-5 cm, 5-10 cm, 10-20 cm, and greater than 20 cm. Non-limiting examples of appropriate thicknesses include 0.05-0.1 millimeters (mm), 0.1-0.2 mm, 0.2-0.3 mm, 0.3-0.4 mm, 0.4-0.5 mm, and greater than 0.5 mm.

In at least one embodiment, any of the devices described herein can have one or more etchings and/or markings thereon to visualize the amount of topical compounds being stored, administered, and/or delivered. Such markings may be in milliliters (mL), cubic centimeters (cc), number of doses, or any combination thereof.

In at least one embodiment, any of the devices described herein may be made of any suitable material for medical and/or cosmetic applications, including one or more plastics, one or more polymers, and the like. Additionally, in at least one example, one or more of the devices described herein are not made of rubber and/or latex to avoid allergic reactions.

At least one embodiment of the disclosure comprises one or more connectors and/or connection mechanisms (e.g., as used on one or more connector ends). Non-limiting examples of such connection mechanisms include, for instance, a luer-style connector, a slip-on connection, a push-on connection, an eccentric tip, a tapered tip, a catheter tip, and combinations thereof. Other different types of connection mechanisms may also be used, non-limiting examples of which include connectors compatible with non-luer-style lock connectors, connectors comprising a plurality of threads without a hub or other protrusion, connectors compatible with non-luer style slip connectors, connectors comprising one or more surfaces for a slip-on connection without threads or other aspects for a screw or twist connection, connectors comprising a plurality of tabs disposed on a surface thereof, connectors comprising a plurality of ribbed aspects, connectors comprising one or more adhesive portions, connectors comprising one or more surfaces that can secure and/or hold, via friction, one or more portions of the delivery device (e.g., any delivery device described herein) in place during delivery and/or administration of the topical compounds, one or more raised sides and/or walls that can secure and/or hold one or more portions of the delivery device (e.g., any delivery device described herein) in place during delivery and/or administration of the topical compounds, one or more transparent windows that can be used to visualize movement of the topical compounds through the device and/or during application and/or administration of the topical compounds, and combinations thereof.

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIGS. 1A-1C show non-limiting examples of syringes, including a syringe with a luer-style connector (FIG. 1C).

FIGS. 2A-2B show a device for applying topical compounds from both a perspective view (FIG. 2A) and an end view (FIG. 2B), according to at least one embodiment of the present disclosure.

FIGS. 3A-3B show non-limiting examples of syringes, including a syringe with a luer-style connector (FIG. 3B), attached to a device for applying topical compounds, according to at least one embodiment of the present disclosure.

FIGS. 4A-4C show portions of a device for applying topical compounds, specifically, a cap (FIG. 4A), a membrane (FIG. 4B), and a combined cap and membrane (FIG. 4C), according to at least one embodiment of the present disclosure.

FIGS. 5A-5C are flow diagrams showing a method for delivering one or more topical compounds, according to at least one embodiment of the present disclosure.

FIG. 6 shows a device for applying and/or delivering topical compounds, the device having a tapered end, according to at least one embodiment of the present disclosure.

FIG. 7 shows a device for applying and/or delivering topical compounds, the device having an eccentric or eccentric-style end, according to at least one embodiment of the present disclosure.

FIG. 8 shows a device for applying and/or delivering topical compounds, the device having an opening and/or port that opens into an interior of the device, according to at least one embodiment of the present disclosure.

FIG. 9 shows a device for applying and/or delivering topical compounds, the device having an angled and/or beveled end, according to at least one embodiment of the present disclosure.

FIG. 10 shows a device for applying and/or delivering topical compounds, the device having an applicator end that narrows from to an elongated and/or domed point, according to at least one embodiment of the present disclosure.

FIG. 11 shows a device for applying and/or delivering topical compounds, the device having an applicator area made, in whole or in part, of a sponge or sponge-like surface, according to at least one embodiment of the present disclosure.

FIGS. 12A-12B show a device for applying and/or delivering topical compounds (FIG. 12A), the device having an applicator end with a plurality of microscopic holes (FIG. 12B), each according to at least one embodiment of the present disclosure.

FIG. 13 shows a device for applying and/or delivering topical compounds, the device having a rounded end, according to at least one embodiment of the present disclosure.

FIG. 14 shows a device for applying and/or delivering topical compounds, the device having a brush end with a plurality of bristles, according to at least one embodiment of the present disclosure.

FIG. 15 shows a device for applying and/or delivering topical compounds, the device having a curved end shaped such that it is ergonomic and/or matches to one or more rounded surfaces on human tissues, according to at least one embodiment of the present disclosure.

FIG. 16 shows a device for applying and/or delivering topical compounds, the device having a dropper and/or dropper-style end, according to at least one embodiment of the present disclosure.

FIG. 17 shows a device for applying and/or delivering topical compounds, the device having an abrasive end with one or more abrasive materials, according to at least one embodiment of the present disclosure.

FIG. 18 shows a device for applying and/or delivering topical compounds, the device having a flat end that can be placed flat against a surface of human tissue, according to at least one embodiment of the present disclosure.

FIG. 19 shows a device for applying and/or delivering topical compounds, the device having an applicator end that has an internal reservoir, according to at least one embodiment of the present disclosure.

FIG. 20 shows a device for applying and/or delivering topical compounds, the device having an applicator end that has the same width as that of a syringe tip and/or the syringe and/or barrel attached to the syringe tip, according to at least one embodiment of the present disclosure.

FIG. 21 shows a device for applying and/or delivering topical compounds, the device having an applicator end that has one or more internal filters, according to at least one embodiment of the present disclosure.

FIG. 22 shows a side view of a connector compatible with non-luer-style lock connectors, according to at least one embodiment of the present disclosure.

FIG. 23 shows a side view of a connector compatible with non-luer-style slip connectors, according to at least one embodiment of the present disclosure.

FIG. 24 shows a side view of a connector having a plurality of tabs disposed on a surface thereof, according to at least one embodiment of the present disclosure.

FIG. 25 shows a side view of a connector having a plurality of ribbed aspects disposed on a surface thereof, according to at least one embodiment of the present disclosure.

FIG. 26 shows a side view of a connector having one or more adhesive portions disposed on a surface thereof, according to at least one embodiment of the present disclosure.

FIG. 27 shows a side view of a connector having one or more surfaces that can secure and/or hold, via friction, one or more portions of a delivery device in place during delivery and/or administration of one or more topical compounds, according to at least one embodiment of the present disclosure.

FIG. 28 shows a side view of a connector having one or more raised sides and/or walls that can secure and/or hold, via friction, one or more portions of a delivery device in place during delivery and/or administration of one or more topical compounds, according to at least one embodiment of the present disclosure.

FIG. 29 shows a side view of a connector having one or more transparent windows, according to at least one embodiment of the present disclosure.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment,” “an embodiment,” “an example embodiment,” etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may effect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms “first,” “second,” etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, “at least one of A, B, and C” indicates A or B or C or any combination thereof. As used herein, the singular forms “a,” “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

Any ranges used herein are in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise,” “comprises,” and “comprising” are to be interpreted inclusively rather than exclusively. Likewise the terms “include,” “including,” and “or” should all be construed to be inclusive, unless such a construction is clearly prohibited from the context. The terms “comprising” or “including” are intended to include embodiments encompassed by the terms “consisting essentially of” and “consisting of.” Similarly, the term “consisting essentially of” is intended to include embodiments encompassed by the term “consisting of.” Although having distinct meanings, the terms “comprising,” “having,” “containing,” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Terms such as, among others, “about,” “approximately,” “approaching,” or “substantially,” mean within an acceptable error for a particular value or numeric indication as determined by one of ordinary skill in the art, which depends in part on how the value is measured or determined. The aforementioned terms, when used with reference to a particular non-zero value or numeric indication, are intended to mean plus or minus 10% of that referenced numeric indication. As an example, the term “about 4” would include a range of 3.6 to 4.4. All numbers expressing dimensions, velocity, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

“Typically” or “optionally” means that the subsequently described event or circumstance may or may not occur, and that the description includes instances where said event or circumstance occurs and instances where it does not.

Wherever the phrase “for example,” “such as,” “including,” and the like are used herein, the phrase “and without limitation” is understood to follow unless explicitly stated otherwise.

The term “administering” or “administration” refers to providing or giving a subject one or more agents, compounds, and/or formulations, either alone or in conjunction with any other compound and/or agent (including, e.g., prophylactic or therapeutic agents), by any effective route. Exemplary routes of administration include, but are not limited to, topical administration, as defined further below.

The terms “agent” or “active agent,” which are used interchangeably herein, refer to a physiologically or pharmacologically active substance that acts locally and/or systemically in a subject's body. An “agent” or “active agent” is a compound or substance that is administered to an individual for the treatment (e.g., therapeutic agent, cancer therapeutic agent, and the like), prevention (e.g., prophylactic agent), or diagnosis (e.g., diagnostic agent) of a disease or disorder. Such agents may also include therapeutics that prevent or alleviate symptoms of a disease or disorder.

The term “compound” refers to a substance formed from one or more chemical elements, arranged together in any proportion or structural arrangement. The one or more chemical elements may be either naturally occurring and/or non-naturally occurring. As used herein, the term “biological compound” refers to a compound of biological origin and/or having one or more effects on a subject's local and/or systemic biological functions. Accordingly, “compounds” or “biological compounds” include, as non-limiting examples, various proteins (e.g., growth factors, hormones, enzymes), nucleic acids, and pharmaceutical products (e.g., drugs, prodrugs). The term “drug” generally refers to a medicine or other substance that has a physiological effect when introduced into a subject. The term “prodrug” generally refers to a biologically and/or chemically inactive compound that can be metabolized by a subject to produce a drug.

The term “connector,” at least as used herein, refers to an article of manufacture for connecting two or more other articles together. Thus, certain portions of a syringe, such as a syringe tip and a needle, may be connected using one or more different types of connectors. Common connectors used in syringes include a “luer-style connector,” a term which, as used herein, encompasses both a lock fitting, in which a tabbed hub on the needle screws into threads on the syringe tip, and a slip fitting, in which the needle and the syringe tip are pressed together, relying on friction (as opposed to screw threads) to hold them together. Other common connectors used in syringes include a slip-on connection, a push-on connection, an eccentric tip, a tapered tip, a catheter tip, and combinations thereof.

The terms “effective amount” or “therapeutically effective amount,” which are used interchangeably herein, refer to the amount of an agent that is sufficient to effect beneficial or desired therapeutic result, including clinical results. An “effective amount” may vary depending upon one or more of: the subject and disease condition being treated, the sex, weight and age of the subject, the severity of the disease condition, the manner of administration, the ability of one or more formulations to elicit a desired response in the subject, and the like. The beneficial therapeutic effect can include, but is not limited to, enablement of diagnostic determinations; prevention of a disease; amelioration of a disease, symptom, disorder, and/or pathological condition; reducing or preventing the onset of a disease, symptom, disorder, and/or pathological condition; and generally counteracting a disease, symptom, disorder, and/or pathological condition. The term “effective amount” includes an amount that is effective to “treat” a subject (e.g., a patient or individual). When a therapeutic amount is indicated, the precise amount of one or more formulations to be administered can be determined by a physician, based on, for instance, considerations such as individual differences in age, weight, one or more symptoms, extent of infection or disease progression, and/or condition of the subject (individual).

The term “membrane” refers to a thin sheet that acts as a boundary, barrier, lining, and/or partition. Membranes may exist naturally inside an organism (e.g., a mammal) or be synthetic/manmade. Thus, the thin sheet may be made of, for instance, tissue or one or more layers of cells. Alternatively, the thin sheet may be made of a manmade material (e.g., one or more resins, one or more polymers such as cellulose and thermoplastics, gauze, and the like). Membranes can further be selectively permeable such that only certain molecules and/or types of molecules may cross the membrane from one side to the other.

The term “molecular weight” generally refers to the relative average chain length of a bulk polymer or protein, unless otherwise specified. In practice, molecular weights can be estimated or characterized using various methods including, for example, gel permeation chromatography (GPC) or capillary viscometry. GPC molecular weights are reported as the weight-average molecular weight (MW), as opposed to the number-average molecular weight (MN). Capillary viscometry provides estimates of molecular weight as the inherent viscosity determined from a dilute polymer solution using a particular set of concentration, temperature, and solvent conditions.

The term “peptide” refers to a polymer of amino acid residues. The amino acid residues may be naturally occurring and/or non-naturally occurring. The terms “polypeptide,” “peptide,” and “protein” are used interchangeably herein. The terms apply to, for instance, amino acid polymers of one or more amino acid residues, an artificial chemical mimetic of a corresponding naturally occurring amino acid, naturally occurring amino acid polymers, and non-naturally occurring amino acid polymers.

The terms “subject,” “individual,” or “patient,” which are used interchangeably herein, refer to a vertebrate, such as a mammal (e.g., a human). Mammals include, but are not limited to, murines (e.g., mice), simians, humans, farm animals, sport animals, and pets. In at least one embodiment, the subject is a non-human mammal, such as a monkey or other non-human primate, mouse, rat, rabbit, guinea pig, pig, goat, sheep, dog, cat, horse, or cow. In at least one example, the subject has a disease or condition that can be treated using one or more agents, formulations, and/or methods (e.g., including the delivery device and/or delivery tip) disclosed herein. In at least an additional example, the subject is a laboratory animal/organism, such as, for example, a mouse, rabbit, guinea pig, or rat. In at least a further example, a subject includes, for instance, farm animals, domestic animals and/or pets (e.g., cats, dogs). In at least a still further example, a subject is a human patient that has a disease or condition, has been diagnosed with a disease or condition, and/or is at risk of having a disease or condition. A “patient” can specifically refer to a subject that has been diagnosed with a particular disease, condition, and/or indication that can be treated with one or more agents, formulations, and/or methods (e.g., including the delivery device and/or delivery tip) disclosed herein.

The term “syringe” refers to a device for intaking and/or expelling one or more agents, compounds, and/or formulations. Generally, a syringe includes the following components: a barrel for holding one or more agents, compounds, and/or formulations to be injected or expelled from the syringe, a plunger that fits inside the barrel and can be pushed or pulled along an inside surface of the barrel, a syringe tip fluidly connected to the barrel and disposed at a front end of the barrel, and a needle that can be attached to the syringe tip or detached from the syringe tip as needed. Certain components of the syringe, including, for instance, the syringe tip and the needle, are connectable and/or connected using a connector, as defined above herein. Common types of syringes include disposable syringes, safety syringes, insulin syringes, hypodermic syringes, multi-shot needle syringes, venom extraction syringes, oral syringes, dental syringes, dose-sparing syringes, glass syringes, plastic syringes (e.g., polyethylene syringes), and combinations thereof.

The term “topical administration” refers to a type of non-invasive administration to one or more tissues (e.g., the skin, orifices, one or more mucosal membranes, and or other tissues) of a subject. Topical administrations can be administered locally; that is, they are capable of providing a local effect in the region of application without systemic exposure. Thus, as a non-limiting example, a topical administration may include application of one or more compounds to a tissue surface. Topical formulations can, additionally, provide one or more systemic effects via, e.g., adsorption into the blood stream of the individual. Routes of topical administration include, but are not limited to, cutaneous and transdermal administration, buccal administration, intranasal administration, inhalation, intravaginal administration, intravesical administration, ophthalmic administration, pulmonary administration, and rectal administration. Accordingly, the term “topical” describes an agent, compound, and/or formulation that may be applied via topical administration.

Further, unless otherwise noted, technical terms are generally used according to conventional usage. Aspects of the disclosure employ, unless indicated specifically to the contrary, conventional methods of chemistry, biochemistry, organic chemistry, molecular biology, microbiology, recombinant DNA techniques, genetics, immunology, and/or cell biology, many of which are described below solely for the purpose of illustration. Such techniques are explained fully in technical literature sources. General definitions of common terms in the aforementioned fields, including, for instance, molecular biology, may be found in references such as, e.g., Krebs et al., Lewin's Genes X, Jones & Bartlett Learning (2009) (ISBN 0763766321); Rédei, Encyclopedic Dictionary of Genetics, Genomics, Proteomics and Informatics (3rd ed.), Springer (2008) (ISBN: 1402067532); Ausubel et al., Current Protocols in Molecular Biology, John Wiley and Sons (updated July 2008) (ISBN: 047150338X); Ausubel et al., Short Protocols in Molecular Biology: A Compendium of Methods from Current Protocols in Molecular Biology (2nd ed.), Wiley-Interscience (1989) (ISBN 0471514705); Glover, et al., DNA Cloning: A Practical Approach, Vol. I-II, Oxford University Press (1985) (ISBN 0199634777); Anand et al., Techniques for the Analysis of Complex Genomes, Academic Press (1992) (ISBN 0120576201); Hames et al., Transcription and Translation: A Practical Approach, Oxford University Press (1984) (ISBN 0904147525); Perbal et al., A Practical Guide to Molecular Cloning (2nd ed.), Wiley-Interscience (1988) (ISBN 0471850713); Kendrew et al., Encyclopedia of Molecular Biology, Wiley-Blackwall (1994) (ISBN 0632021829); Meyers et al., Molecular Biology and Biotechnology: A Comprehensive Desk Reference, Wiley-VCH (1996) (ISBN 047118571X); Harlow et al., Antibodies: A Laboratory Manual, Cold Spring Harbor Laboratory Press (1988) (ISBN 0879693746); Coligan et al., Current Protocols in Immunology, Current Protocols (2002) (ISBN 0471522767); Annual Review of Immunology; articles and/or monographs in scientific journals (e.g., Advances in Immunology); and other similar references.

Embodiments of the disclosure comprise devices, methods, and/or systems that permit a user to apply and/or deliver topical compounds (e.g., topical medications) to the surface of mammalian tissue (e.g., skin, mucous membranes, and/or other tissue surfaces). At least some such embodiments comprise a device that is connectable to one or more types of known syringes.

A skilled artisan will appreciate that known syringes generally include the following components: a barrel for holding one or more compounds to be injected or expelled from the syringe, a plunger that fits inside the barrel and can be pushed or pulled along an inside surface of the barrel, a syringe tip fluidly connected to the barrel and disposed at a front end of the barrel, and a needle that can be attached to the syringe tip or detached from the syringe tip as needed.

It will further be appreciated that known syringe tips have different kinds of connectors and/or connection mechanisms to connect the syringe tip with the needle. Generally, such connectors are small-scale and/or small bore fittings (e.g., where the size of the connector opening is less than about 8.5 millimeters) for making leak-free connections. One non-limiting example of such a connector and/or connection mechanism is a “luer-style connector,” a term which, as used herein, encompasses both a lock fitting, in which a tabbed hub on the needle screws into threads on the syringe tip, and a slip fitting, in which the needle and the syringe tip are pressed together, relying on friction (as opposed to screw threads) to hold them together. Further, the term “luer-style connector” as used herein is not limited to any specific dimensions, name brands, or manufacturers.

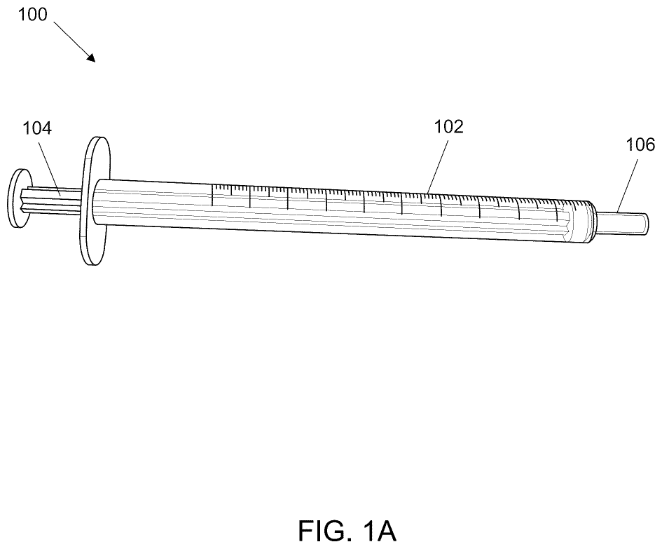

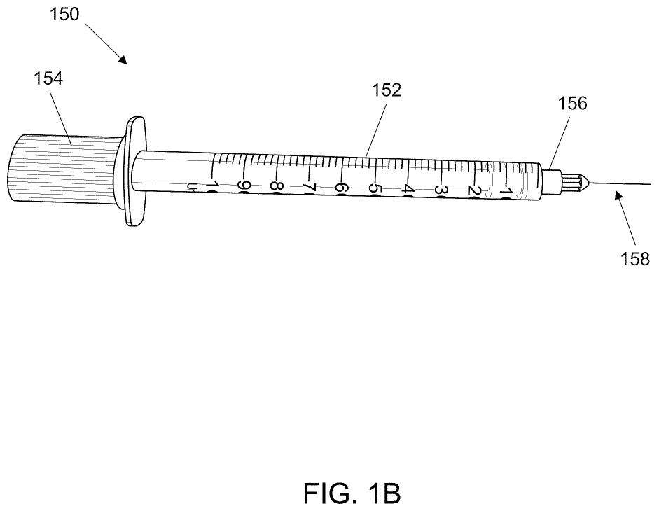

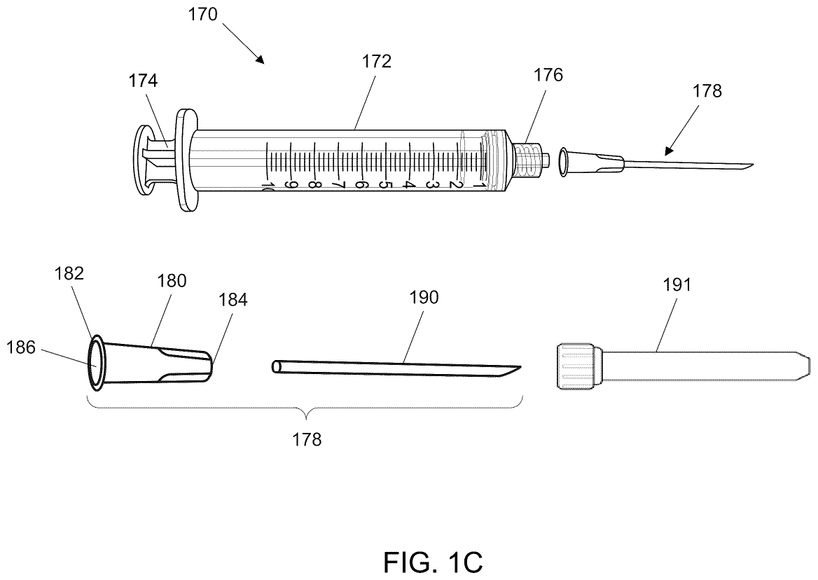

Turning now to FIGS. 1A and 1B, two non-limiting examples of known syringes are shown. FIG. 1A shows a syringe 100 that has a barrel 102, a plunger 104, and a syringe tip 106. The needle is not shown. FIG. 1B shows a syringe 150 that has a barrel 152, a plunger 154, a syringe tip 156, and a needle 158 connected to the syringe tip.

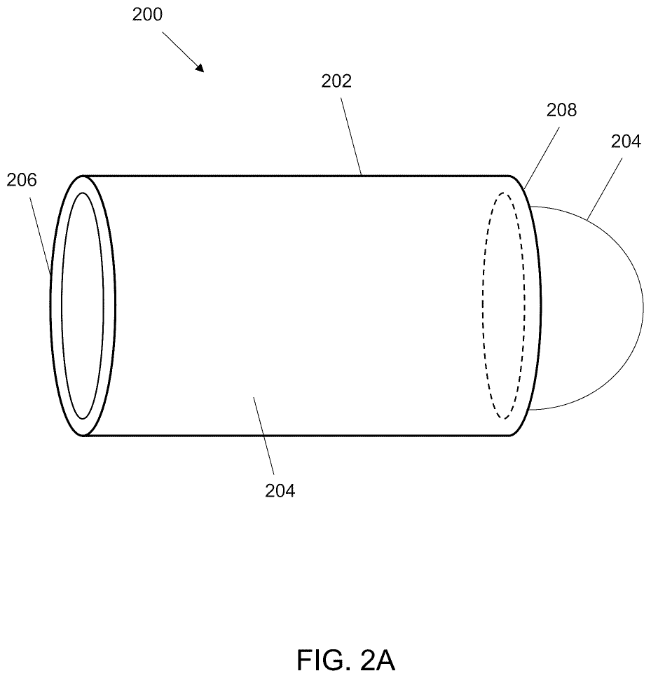

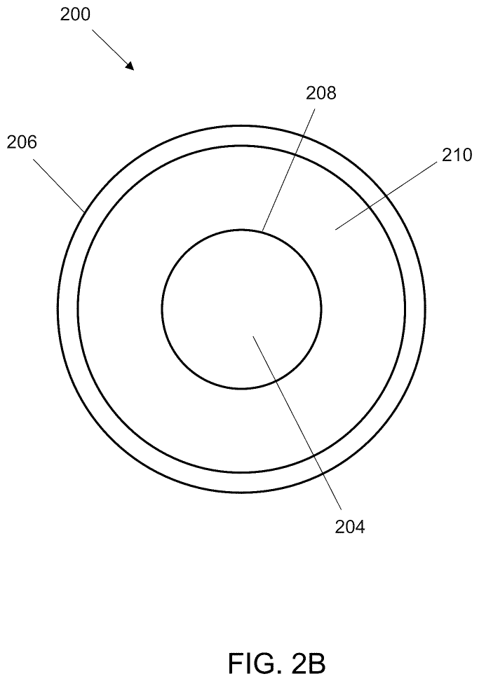

FIG. 1C shows another non-limiting example of a syringe with a luer-style connector. Specifically, a syringe 170 has a barrel 172, a plunger 174, and a syringe tip 176. The syringe tip 176 utilizes a luer-style connector, as described above herein. The needle 178 is shown in a position detached from, and unconnected to, the syringe tip 176. Thus, in at least one embodiment, a device for applying and/or delivering topical compounds (e.g., as shown in FIGS. 2A and/or 2B) may be attached to the syringe tip 176 in lieu of the needle 178.

Also shown in FIG. 1C is an exploded view of the needle 178. The needle 178 includes a hub 180 and a needle tip 190, in addition to an optional cover 191 for covering and/or capping the needle tip. It should be appreciated that other needles disclosed herein (e.g., needle 158) also include a hub, a needle tip, and/or a cover. Accordingly, the description of the needle 178 presented herein is not limited solely to that shown in FIG. 1C. Generally, a “hub” or “needle hub,” terms which are used interchangeably herein, is a hollow structure that is configured to engage on one end with a syringe and on another end with the needle tip. Accordingly, the hub 180 is hollow with an inner area 186 that includes a flange 182 that is configured to engage with, and/or attach to, the syringe 170 and, in particular, the syringe tip 176. During use, one or more compounds in the barrel 172 move through the syringe tip 176 and into the inner area 186, before being ejected through the needle tip 190. The needle tip is connectable with, and/or connected to, the hub 180 at a needle tip end 184. Such needle tip end is disposed at one end of the hub 180, e.g., an end distal to the syringe, while the flange 182 is disposed at an opposite end of the hub, e.g., an end proximal to the syringe. The flange 182 is configured to attach the hub 180 and/or the needle tip 190 to the syringe 170 via the syringe tip 176. The flange 182 may be a generally circular structure that engages with, and/or attaches to, the syringe tip 176 via one or more connectors and/or connection mechanisms (e.g., a luer-style connector). Thus, for instance, a lock fitting luer-style connector may be used in which the flange 182 contains one or more tabs that screw into one or more threads on the syringe tip 176. Additional connectors and/or connection mechanisms, as described above herein, may also be used, such as, for instance, a slip-on connection, a push-on connection, an eccentric tip, a tapered tip, a catheter tip, and combinations thereof.

Further variations on the structure of the hub 180 are possible. For instance, in at least one embodiment, one or more extendable sections, fins, and/or grips may be disposed on an outer surface of the hub to assist the user in gripping the hub. In at least another embodiment, the needle tip end 184 may contain one or more structures (e.g., dimples, protrusions, and/or indentations) to assist in engagement and/or connection of the hub 180 with the needle tip 190. In at least a further embodiment, the flange 182 contains one or more marks to assist a user in identifying one or more properties of the syringe and/or needle being used (e.g., syringe type, syringe brand, needle type, needle brand, needle gauge, syringe measurements, including barrel volume measurements, etc.). Non-limiting examples of such marks include color-coding, text, logos, indentations, protrusions, ridges, and the like. In any one or more of the embodiments mentioned herein, the hub 180, and/or one or more portions thereof, may be composed of one or more different materials, including, for instance, one or more plastic polymers (e.g., polypropylene, polyethylene, polycarbonate, thermoplastic elastomers, terephthalate, etc.), one or more resins (e.g., polyactide, starch-filled polypropylene, polyhydroxyalkanoates, etc.), and the like.

The cover 191 is used for covering the needle tip 190. To ensure that the cover does not fall off, one or more connectors and/or connection mechanisms can be used, including any of those described herein. For instance, in at least one embodiment, the cover includes one or more snap-on connections. In at least another embodiment, the cover includes one or more slip-on connections. The cover 191 can perform one or more functions related to the syringe 170, the hub 180, and/or the needle 190, and/or any uses thereof. Such one or more functions include, for instance, preventing the needle tip from being bent or damaged, preventing the needle tip from being partially or completely clogged, preventing the needle tip from injuring or harming (e.g., pricking) a user and/or any other individual (e.g., a patient or subject), preventing one or more compounds and/or formulations in the syringe (e.g., in the barrel) from escaping or leaking out, shielding one or more portions of the syringe (e.g., the needle) from one or more environmental effects (e.g., oxidative effects, corrosive effects, bleaching effects, etc.), shielding one or more portions of the syringe (e.g., the needle) from one or more light wavelengths (e.g., wavelengths below 500 nanometers (nm), wavelengths between 300-500 nm, wavelengths between 290-500 nm, wavelengths between 290-450 nm, wavelengths above 500 nm, wavelengths from 500-800 nm, etc.), and the like. Protection from one or more light wavelengths may be especially important when using the syringe to apply one or more light-sensitive compounds such as, for example, aminophylline, chlorpheniramine maleate, chlorpromazine hydrochloride (HCl), cisplatin, dacarbazine, diazepam, diazoxide, digoxin, diphenhydramine, dopamine HCl, doxycycline hyclate, droperidol, epinephrine HCl, fluorouracil, folic acid, furosemide, haloperidol, hydrocortisone, isoproterenol, levarterenol bitartrate, menadiol sodium diphosphate, methadone, morphine, nitroglycerin, nitroprusside solution, phenylephrine HCl, phytonadione, promethazine, propranolol HCl, streptomycin, testosterone, triflupromazine HCl, vinblastine, vincristine, one or more vitamins (e.g., vitamin B), and the like. The cover 191 may also perform one or more functions related to applying the one or more compounds and/or formulations in the syringe. As a non-limiting example, the cover 191 may store and/or apply one or more secondary compounds that are applied to a subject and/or patient in addition to the one or more compounds and/or formulations in the barrel of the syringe. Such secondary compounds include, for instance, one or more antimicrobial compounds including antibacterial compounds, antiviral compounds, and/or antifungal compounds (e.g., coumarin, cinnamic acid, vancomycin, hypericin, iturin-like mycosubtilin, bacillomycin, subtulene A, eumycin, antiviral drugs, clotrimazole, miconazole, ketoconazole, etc.), one or more adjuvants (e.g., aluminum hydroxide, aluminum phosphate, calcium alginate, etc.), one or more dressings and/or coverings (e.g., wound coverings), one or more grafts (e.g., skin grafts or any other type of graft described herein), and the like.

The cover 191 may, in at least one embodiment, be adapted to fit one or more hubs and/or needle tips (e.g., the hub 180 and/or the needle tip 190) such as, for example, needle tips of 51 millimeters (mm)+1 mm in length and associated hubs, needle tips having a length of less than 1 inch (e.g., ½ inch) and associated hubs, needle tips having a length of more than 1 inch (e.g., 1.5 inches) and associated hubs, needle tips of 10-304 mm and associated hubs, metal needle tips and associated hubs, Kel-F needle tips and associated hubs, standard removable needle tips and associated hubs, small gauge removable needle tips and associated hubs, needle tips having one or more gauges between 7 and 33 and associated hubs, and the like. In at least an additional embodiment, the cover 191 is adapted to evacuate one or more materials from one or more portions of the syringe (e.g., liquids, gases, gels, etc.). In at least an additional embodiment, the cover 191 is filled, either entirely or partially, with one or more materials (e.g., inert gases such as helium, argon, krypton, neon, radon, and/or any other noble gas). In at least another embodiment, the cover 191 is configured to fit with, attach on to, and/or connect with, one or more additional hubs (e.g., secondary hubs, tertiary hubs, etc.) associated with one or more additional medical tools and/or medical devices (e.g., additional syringes, drip and/or intravenous (IV) tubes, drip and/or IV bags, tube drippers, infusion apparatuses, cannulas, injectors, beakers, flasks, test tubes, suction tubing, etc.).

As with the hub 180, the cover 191 may be composed of one or more materials, including any one or more of the materials disclosed herein from which the hub 180 are composed. Accordingly, the cover 191 can include one or more plastic polymers (e.g., polypropylene, polyethylene, polycarbonate, thermoplastic elastomers, terephthalate, etc.), one or more resins (e.g., polyactide, starch-filled polypropylene, polyhydroxyalkanoates, etc.), and the like.

In at least one embodiment of the disclosure, a device for applying and/or delivering topical compounds can be engaged and/or attached to a syringe via a syringe tip (e.g., tip 176). A non-limiting example of such a device 200 is shown in FIG. 2A in a perspective view. The device 200 comprises a cap 202 and a membrane 204. The cap 202, which may be hollow in at least one embodiment, comprises a first end, which is the open connector end 206, for engaging and/or attaching (e.g., via a releasable connection) to a syringe (e.g., the syringes 100 and/or 150) via the syringe tip (e.g., the syringe tips 106 and/or 156). The cap 202 further comprises a second end, which is the delivery end 208, disposed opposite to the connector end 206 and fluidly connected to the connector end 206. The cap additionally comprises a cap body 210 disposed between, and connecting, the connector end 206 and the delivery end 208.

The delivery end 208 is further connected to, and may be attached to and/or affixed to, the membrane 204. Such connection may be made by any known mechanism, including, for instance, inserting one end of the membrane into the delivery end.

The connector end may comprise, in at least one embodiment, a luer-style connector. In at least another embodiment, the connector end may comprise any other known connection mechanism, including, for instance, any small-scale and/or small bore fitting for making a leak-free connection (e.g., any fitting for medical devices and/or medical accessories governed by International Organization for Standardization (ISO) standard 80369 or any other similar recognized standard). Non-limiting examples of such connectors and/or connection mechanisms include, for instance, a slip-on connection, a push-on connection, an eccentric tip, a tapered tip, a catheter tip, and combinations thereof. Thus, in at least one embodiment, the device is compatible with, and may be engaged and/or attached to, any syringe tip having one or more of the aforementioned connection mechanisms. The device may further be compatible with, and may be engaged and/or attached to, a tip for any known type of syringe, including, for instance, a disposable syringe, a safety syringe, an insulin syringe, a hypodermic syringe, a multi-shot needle syringe, a venom extraction syringe, an oral syringe, a dental syringe, a dose-sparing syringe, a glass syringe, a plastic syringe (e.g., a polyethylene syringe), and combinations thereof.

The device 200 is further shown in FIG. 2B in a view from the open connector end 206. An interior surface of the cap body 210 is shown, as is an interior surface of the membrane 204 that is connected to the delivery end 206.

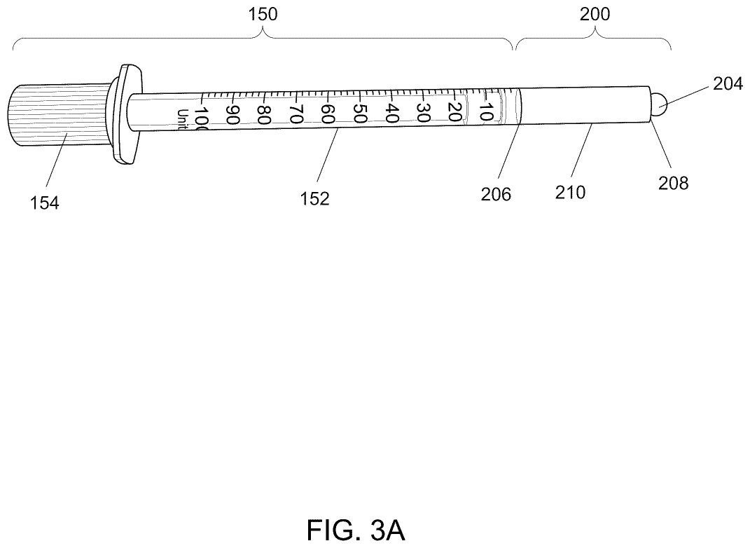

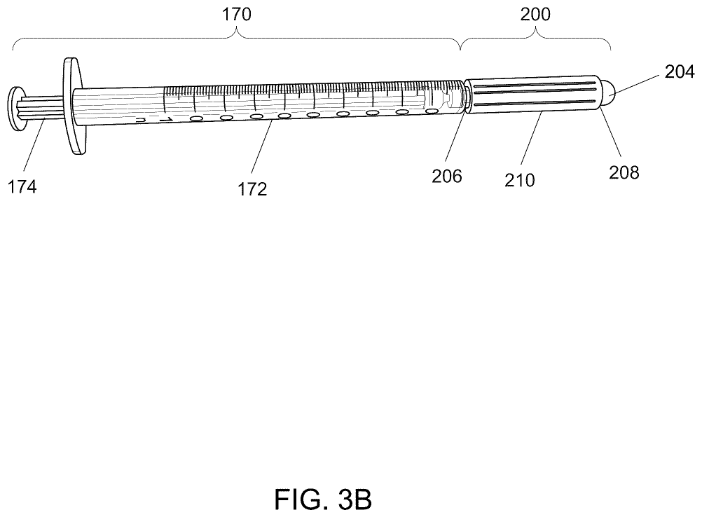

Turning now to FIG. 3A, a device for applying and/or delivering topical compounds, specifically the device 200, is shown connected to a syringe, specifically the syringe 150, via a syringe tip. It should be appreciated that the syringe tip in FIG. 3A is not visible because the device 200, and specifically the connector end 206, is engaged with the syringe tip (e.g., via a releasable connection) such that the device covers the syringe tip and obscures it from view. Further, the connector end 206 is engaged with the syringe tip such that the connector end is fluidly connected to the syringe tip and/or one or more openings in the syringe tip. Thus, as a non-limiting example, the connector end 206 may comprise a luer-style connector that is a lock fitting, in which the connector end screws into threads on the syringe tip, thereby obscuring the syringe tip from view.

When the device is so engaged and/or attached to the syringe tip, one or more compounds (e.g., topical medications) inside the syringe can be introduced from the barrel (e.g., the barrel 152) through the syringe tip (e.g., the syringe tip 156) and through the device, and specifically through the membrane (e.g., the membrane 204) attached to the delivery end of the device, to a surface of a tissue (e.g., skin).

Similarly, FIG. 3B shows the device 200 connected to another non-limiting example of a syringe, specifically the syringe 170, via a syringe tip that utilizes a luer-style connector. Again, it should be appreciated that the syringe tip in FIG. 3B is not visible because the device 200, and specifically the connector end 206, is engaged with the syringe tip (e.g., via a luer-style connector) such that the device covers the syringe tip and obscures it from view. Further, the connector end 206 is engaged with the syringe tip, which is a luer-style connector with either a lock fitting or a slip fitting, such that the connector end is fluidly connected to the syringe tip and/or one or more openings in the syringe tip. Accordingly, the connector end 208 obscures the syringe tip from view.

Thus, in at least one embodiment, the device 200 may be used with the syringe 170. When the device is so engaged and/or attached to such a syringe, one or more compounds (e.g., topical medications) inside the syringe can be introduced from the barrel (e.g., the barrel 172) through the syringe tip (e.g., the syringe tip 176, shown in FIG. 1C) and through the device, and specifically through the membrane (e.g., the membrane 204) attached to the delivery end of the device, to a surface of a tissue (e.g., skin).

In at least one embodiment, the membrane is selectively permeable such that the membrane permits the one or more compounds inside the syringe to move through the membrane. It should be appreciated that these selectively permeable properties can be selected based on the biological, chemical, and/or physical nature of the one or more compounds to be delivered to the surface of the tissue. Non-limiting examples of membrane materials include, for instance, one or more polymers (including synthetic/manmade polymers), one or more resins, etc. Thus, the membrane may be composed of, for instance, cellulose, gauze, and the like.

The one or more compounds may be one or more topical medications or other compounds, in any formulation or concentration, including, for instance, triamcinolone, fluorouracil (also known as “5-FU”), tranexamic acid (also known as “TXA”), azelaic acid, one or more bleaching agents (e.g., hydroquinone), one or more topical numbing agents (e.g., lidocaine, bupivacaine), one or more topical antibiotics, one or more dyes or stains (e.g., bonnet blue, gentian violet), one or more topical anti-inflammatory compounds such as nonsteroidal anti-inflammatory drugs (e.g., diclofenac, ibuprofen, Toradol), vitamins (e.g., vitamin A, vitamin B12, vitamin E, vitamin K), one or more topical steroids, one or more topical darkening agents for conditions such as hypopigmented scars (e.g., bimatoprost), anti-oxidants (e.g., N-acetyl-cysteine, glutathione), one or more micro-dosed compounds (e.g., botulinum toxin, hyaluronic acid, other compounds used in micro-needling reservoir treatments), one or more skincare compounds, one or more cosmetic compounds, one or more nutraceutical compounds, and combinations thereof.

Further non-limiting examples of the one or more compounds that could be administered, topically and/or otherwise, through the embodiments described herein include one or more processed human-derived components (e.g., nanofat, platelet-rich plasma (PRP), autogenous materials, and/or allograft materials), one or more types of exosomes, and the like. Each of these will be described briefly below.

Nanofat, in at least some applications, is a bundle of adipose tissue-derived stem cells. Adipose tissue generally contains a heterogenous population of cells, including mesenchymal stromal cells. Adipose-derived cells, including stem cells, can be more easily extracted than other types of stem cells. Further, adipose tissue contains a variety of proteins, such as, for instance, growth factors, transcriptional factors, cytokines, and the like. Accordingly, adipose tissue-derived products can exhibit anti-inflammatory, angiogenesis, and/or immune modulating effects. Such products include, for example, nanofat, microfat, adipose-derived stem cells (“ASC” or “ASCs”), and exosomes, which are described later herein.

Nanofat itself can be produced by, for instance, emulsification and filtration of aspirates from adipose tissue. These aspirates are then physically broken down and/or disintegrated to obtain a product that can be administered to a subject (e.g., via injection, micro-needling, intradermal, subcutaneous, local infiltration, etc.). Generally, nanofat formulations do not contain mature adipocytes (fat cells), but do contain ASCs, various growth factors (e.g., vascular endothelial growth factor (VEGF), platelet-derived growth factor (PDGF), hepatocyte growth factor (HGF), transforming growth factor-beta (TGF-β), and the like), and cytokines (e.g., interleukin (IL)-1RA, IL-4, IL-8, IL-10, IL-13, and the like). Nanofat can therefore be used in regenerative medicine treatments in a variety of medical fields, such as, for example, plastic surgery, dermatology, orthopedics, and the like. In at least one embodiment, the devices, applicators, and/or methods described herein are used to administer one or more formulations containing nanofat for one or more medical treatments, such as, for example, treatment of burns, plastic surgery treatments (e.g., scar reduction), dermatological treatments (e.g., wrinkle reduction, correction of dark eye circles), orthopedic treatments (e.g., regeneration of degenerated and/or diseased cartilage, tendon, and/or ligaments), and combinations thereof.

PRP, which may also be referred to as autologous conditioned plasma, is a product derived from whole blood, which is centrifuged to remove red blood cells. PRP is therefore a concentrate of plasma protein that contains platelets (which may be at, for instance, a concentration 3-5 times that of normal physiological levels), growth factors, cytokines, and other peptides that can stimulate injury repair, tissue healing, wound healing, and tissue rejuvenation. Similar products include, for example, platelet-rich fibrin (PRF). Without wishing to be bound by theory, the concentration of platelets in PRP at a wound or injury site may increase the rate of healing by increasing the rate of tissue repair and/or proliferation. Accordingly, PRP and/or PRF can be used in various medical fields and/or medical treatments, including, for example, dermatology (e.g., skin treatments), orthopedics (e.g., treating muscle strains, tendon injuries, joint injuries, and soft tissue injuries), plastic surgery (e.g., face lifts), and the like. In at least one embodiment, the devices, applicators, and/or methods described herein are used to administer one or more formulations containing PRP and/or PRF for one or more medical treatments, including those described above herein.

Similarly, the devices, applicators, and/or methods described herein may be used to administer one or more formulations containing one or more peptides (e.g., glycoproteins, growth factors such as PDGF and/or TGF-β, cytokines, etc.) and/or one or more cellular materials. Such peptides may be, for instance, derived from PRP or from other human-derived sources (e.g., bone marrow cells). Purely as a non-limiting example, PDGF and TGF-ß can participate in one or more injury repair, tissue healing, and/or tissue regeneration processes. Non-limiting examples of the aforementioned one or more cellular materials include one or more types of cells (e.g., adipose cells, blood cells, endothelial cells, epithelial cells, fibroblasts, stem cells, progenitor cells, muscle cells, macrophages and/or other immune cells, etc.), one or more cells that have been genetically engineered and/or modified, bone marrow, lipids, liposomes, vesicles, and combinations thereof.

Further, the devices, applicators, and/or methods described herein may be used to administer one or more formulations containing one or more autogenous materials and/or one or more allograft materials. An “autogenous material” is a material that is taken from the subject or patient's own body. An “allograft material” is a material taken from an individual of the same species as the subject or patient. Both autogenous and allograft materials can be used in a variety of medical treatments, including grafts (e.g., dental grafts, bone grafts). Thus, autogenous and/or allograft materials can be administered to assist in tissue repair, wound healing, and other similar applications. For instance, such materials can be used for treating surgical wounds, deep wounds that include damage of the dermis, eye wounds, dental and oral cavity wounds, internal ulcers (e.g., diabetic, dermal, arterial, venous, and other types of ulcers), burns, scars, and the like.

Additionally, as mentioned above herein, autogenous and/or allograft materials can be used for one or more types of grafts. In at least one embodiment, the devices, applicators, and/or methods described herein may be used to administer a graft. One specific non-limiting example is a skin graft over an open wound, burn, and/or scar. Non-limiting examples of skin and other grafts include autologous skin grafts, allografted skin, artificial skin, autodermic grafts, autoepidermic grafts, dermic grafts, epidermic grafts, avascular grafts, bone grafts, blepharoplastic grafts, cutis grafts, delayed grafts, fascia grafts, full thickness grafts, heterologous grafts, homologous grafts, xenografts, lamellar grafts, mesh grafts, mucosal grafts, patch grafts, pedicle grafts, penetrating grafts, split skin grafts, thick split grafts, and the like.

In at least an additional embodiment, the devices, applicators, and/or methods described herein may be used to administer one or more formulations containing one or more exosomes and/or one or more different types of exosomes. The aforementioned exosomes may be used to treat one or more diseases or conditions; accordingly, the number of exosomes in the one or more formulations can be maximized to be, for instance, 90%, 80%, 70%, 60%, 50%, 40%, or more than 30%. In at least one example, high concentrations of exosomes can be used to treat one or more diseases or conditions (e.g., cancers, tumors, and the like). Such high concentrations can be produced using, for instance, induction of exosomes from stem cells via treatment of these stem cells with cytokines, treatment with liposome stimulation using one or more stimulant liposomes (e.g., neutral or cationic liposomes, see Emam et al., Biol. Pharm. Bull. 2018; 41(5):733-742), and/or other physical and/or biological methods previously described. See, e.g., Phan et al., J. Extracell. Vesicles. 2018; 7(1): 152223. In at least a further example, the one or more types of exosomes include isolated exosomes, which are exosomes that are physically separated from their natural environment(s). As a non-limiting example, an isolated exosome may be separated from any cells or tissue with which it is naturally associated, including, for instance, various types of stem cells. In at least another example, the one or more types of exosomes include exosomes that are engineered in vitro. This can be achieved through, for example, modifying the cell and/or tissue with which the exosome is naturally associated, exposing such cells and/or tissue to one or more stimulus molecules and/or compounds, and the like.

In at least additional examples, the exosomes administered contain one or more biological compounds. Such compounds may be at a higher level or concentration than the level or concentration present in a naturally occurring exosome (e.g., about 1.5-fold higher, about 3-fold higher, about 10-fold higher, about 20-fold higher, about 50-fold higher, about 100-fold higher, or about 200-fold higher). Non-limiting examples of these compounds include any peptide, any nucleic acid, any lipid, any carbohydrate, osteoinductive factors (e.g., any factor that promotes healing and/or development of bone), neuronal regeneration factors (e.g., any factor that promotes healing and/or regeneration of neurons), immunomodulatory factors (e.g., any factor that influences and/or modulates one or more immune responses and/or immune pathways), extracellular matrix binding factors (e.g., any factor present in the extracellular matrix), extracellular matrix components (e.g., collagen, elastin, fibrin, one or more glycoproteins, one or more proteoglycans, one or more polysaccharides), one or more polymers (e.g., one or more biopolymers), one or more growth factors (e.g., transforming growth factors, platelet-derived growth factors), and one or more micro-RNAs (miRNAs) (e.g., miRNA 218, miRNA 9-5p, miRNA 19a-3p, miRNA 30a-5p, miRNA 212-5p, miRNA 323-5p, miRNA 15a, miRNA 15b, miRNA 16, miRNA 424, miRNA 497, etc.).

One or more compounds described herein can be used to treat a variety of conditions, including, for instance, skin conditions such as scars, burns, hyperpigmentation, inflammation, and/or fibrosis. Purely as non-limiting examples, the device could be used to deliver (1) topical steroids to treat scars, (2) growth factors and anti-scarring medications to treat burns, (3) anti-aging compounds and/or vitamins to improve skin texture, quality, and/or clarity, (4) anti-inflammatory compounds to prevent and/or treat inflammation, and (5) medication or other treatment compounds as part of skin laser treatments, microneedling, dermal abrasion, and the like.

It should also be appreciated that the device may be used to apply one or more topical compounds to mammalian tissues and/or areas other than the skin. For instance, the device can be used to apply one or more compounds (e.g., fibrin-based glues) to nerve conduits, the outside of micro-vascular anastomosis, the outside of vein grafts, allografts, and/or other grafts, the outside of tendons, acellular dermal matrices, the outside of bone grafts, bone reconstructions, and/or bone substitutes, the outside of the intersections of bone and/or peel and bone substitutes, the outside of bioengineered interfaces and/or bioengineered devices, the outside of implanted devices, and combinations thereof.

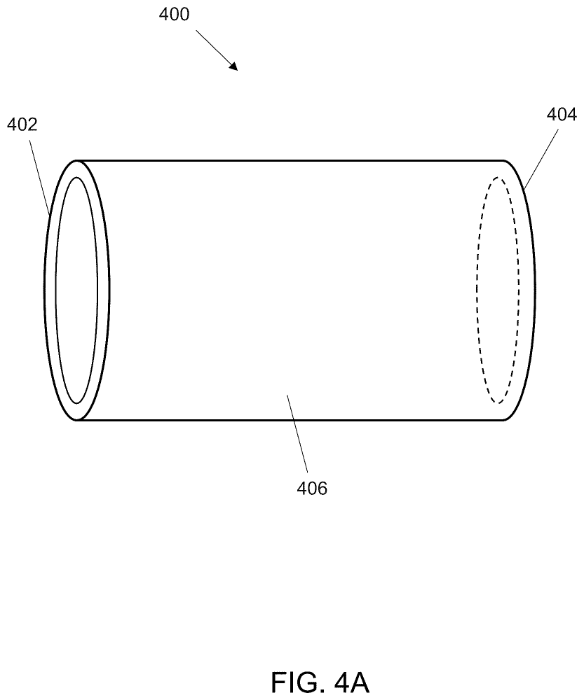

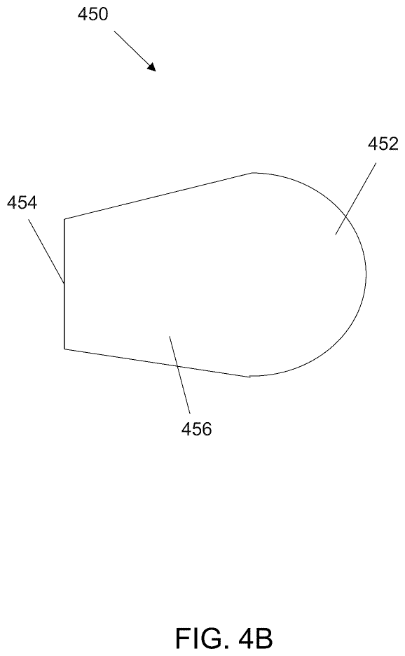

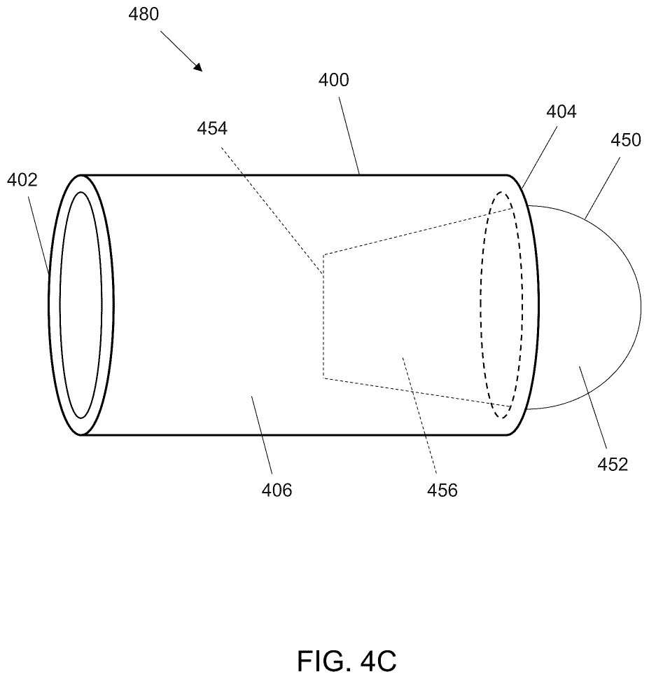

In at least a further embodiment, the device can be detachable and/or separable into individual portions, such as, for instance, a cap and a membrane. Thus, FIGS. 4A-4B show a cap 400 (FIG. 4A) that is detachable and/or separate from a membrane 450 (FIG. 4B). The cap 400 may have the same or similar construction as, for instance, the cap 202. The cap 400 comprises an open connector end 402, an open delivery end 404 that is fluidly connected to the open connector end 402, and a cap body 406 disposed between, and connecting, the connector end and the delivery end. It should be appreciated that, in at least one embodiment, the open delivery end 404 can be covered and/or closed with, for instance, a membrane (e.g., the membranes 204, 450).

Turning now to FIG. 4B, the membrane 450 has an outer and/or closed end 452 through which one or more topical compounds can be delivered, an open inner end 454 through which the one or more topical compounds can be introduced into the membrane, and a neck portion 456 connecting the outer and the open inner end. In at least one embodiment, the open inner end 454 of the membrane 450 can be inserted into the open delivery end (e.g., the delivery ends 208, 404) of a cap, such that the open inner end 454 is fluidly connected to the delivery end. Thus, one or more topical compounds introduced into the cap (e.g., when the cap is connected to a syringe tip) flows through the cap from the connector end (e.g., the connector ends 206, 402) to the delivery end and into the membrane via the open inner end 454. The one or more compounds then flow through the neck portion and are extruded out from the outer end 452 on to a surface of mammalian tissue (e.g., skin, mucous membranes, and/or other tissue surfaces).

In at least one embodiment, the open inner end 454 and/or the neck portion 456 are attached to an inner surface of the cap. In at least a further embodiment, the open inner end and/or the neck portion are simply inserted, in a removable fashion, into the cap via the open delivery end (e.g., the delivery ends 208, 404).

In at least a further embodiment, the membrane 450 can be inserted and/or connected to the cap 400, resulting in a combined cap-membrane device 480, as shown in FIG. 4C. The cap 400 comprises an open connector end 402, a delivery end 404, and a cap body 406. Both the open inner end 454 and the neck portion 456 of the membrane 450 are inserted into the delivery end 404, such that the outer end 452 of the membrane is disposed outside of the delivery end 404.

Thus, like the device 200, the combined cap-membrane device 480 may be engaged and/or attached to a syringe tip (e.g., any of the types of syringe tips described above herein). Specifically, the open connector end 402 can be engaged and/or attached (e.g., via a releasable connection) to such a syringe tip. One or more compounds (e.g., topical medications) inside the syringe can be introduced from the barrel (e.g., the barrels 102, 152) and flow through the syringe tip (e.g., the syringe tips 106, 156) and through the connector end into the cap. The one or more compounds can then flow through the open inner end 454 of the membrane 450, through the neck portion 456, and finally through the outer end 452. The portion of the one or more compounds extruded through the outer end 452 can then be topically applied and/or delivered to the surface of mammalian tissue (e.g., skin, mucous membranes, and/or other tissue surfaces). These one or more compounds may be any of the one or more topical compounds described above herein.

A user (e.g., a medical professional) of at least one embodiment of the device (e.g., the device 200, the combined cap-membrane device 480) can apply the one or more compounds inside the syringe (e.g., the syringes 100, 150) to the surface of tissue by holding the syringe as a pen or pencil, and exerting force on an outside surface of the syringe barrel (e.g., the barrels 102, 152) to move the one or more compounds through the device, and specifically through the membrane (e.g., the membranes 204, 450) attached to the delivery end of the device, to the surface of the tissue. Thus, as a non-limiting example, the aforementioned user could hold the syringe with his or her thumb, forefinger, and/or middle finger.

It should be appreciated that such manner of holding the syringe is in contrast to known methods, in which the user's thumb contacts a top surface of the plunger of the syringe, rather than the syringe barrel, in order to exert force on the plunger to depress the plunger into the barrel, thereby ejecting the one or more compounds inside the barrel through a needle attached to the syringe tip.

It should further be appreciated that holding the syringe as one would a pen or pencil may provide several advantages, especially with respect to the topical application of the one or more compounds, including, for instance, more and/or better control of the amount of the one or more compounds applied, limiting the amount of the one or more compounds applied, more and/or better control of the surface area to which the one or more compounds are applied, prevention of overmedication, prevention of waste, and/or cost savings from avoiding excessive application amounts of the one or more compounds.

In at least one embodiment, the membrane of the device (e.g., the device 200, the combined cap-membrane device 480) is additionally pre-coated with one or more topical agents that can be activated by one or more known mechanisms (e.g., via a secondary agent, ultraviolet (UV) light, and the like). A non-limiting example of such topical agents are caged compounds. Generally, caged compounds are compounds that have a photolabile protecting group attached to a molecule or functional group, thereby rendering the entire compound biologically inert. The caged compound can be activated by exposure to one or more types of irradiation (e.g., UV light), which removes the protecting group and releases the molecule or functional group. For instance, nitric oxide compounds can be caged such that, upon exposure to UV light, the nitric oxide (NO) is released. NO may influence and/or participate in a variety of biological processes, including immune responses. NO can also act as an antibacterial and/or antiviral compound by, for instance, rupturing the plasma membrane of bacteria and/or viruses. Accordingly, in at least one embodiment, the membrane of a device as described herein is pre-coated with a caged compound (e.g., a caged nitric oxide compound) that is then activated by exposure to UV light. In the case of a caged nitric oxide compound, NO is then released, which can then be administered to a subject.

In at least a further embodiment, one or more devices described herein can be used to administer one or more caged compounds that release a primary medicament and/or a secondary medicament in a temporally controlled fashion via controlled exposure to UV light. Such medicaments, which can be compounds that are used for one or more medical treatments, include, for example, drugs, prodrugs, signaling molecules, secondary messengers, neurotransmitters, inositols, calcium, capsaicin, nucleosides, nucleotides, peptides, enzymes, nucleic acids, antibacterial compounds, antiviral compounds, and the like.

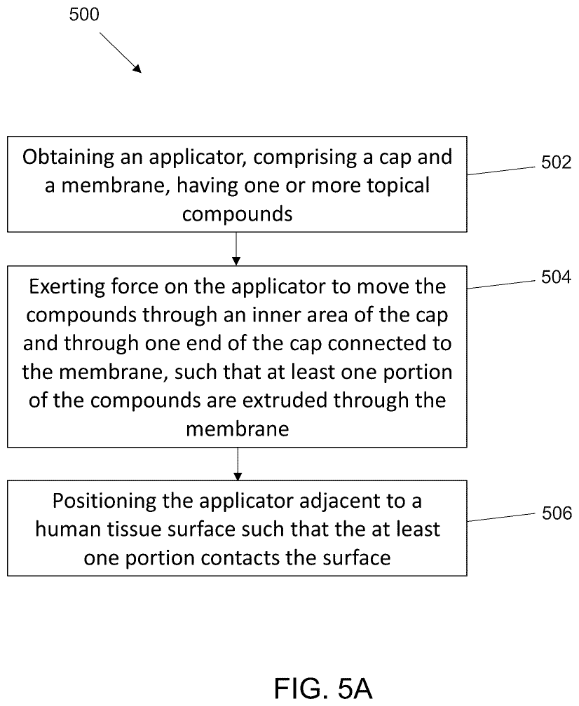

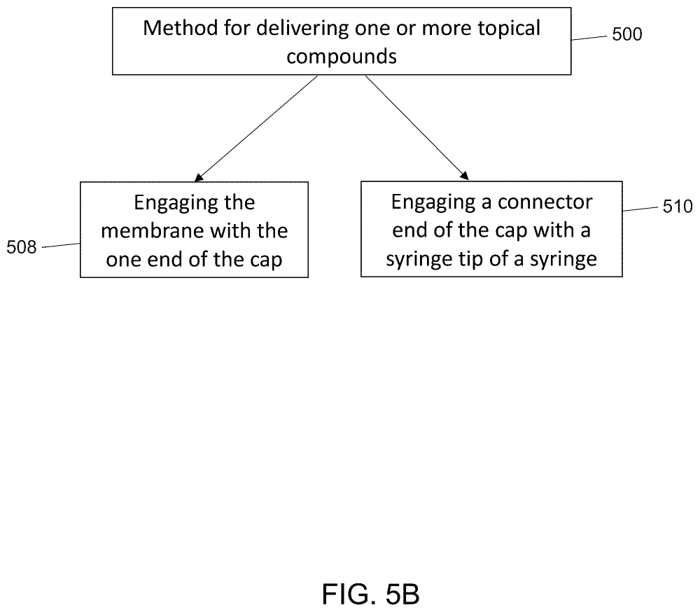

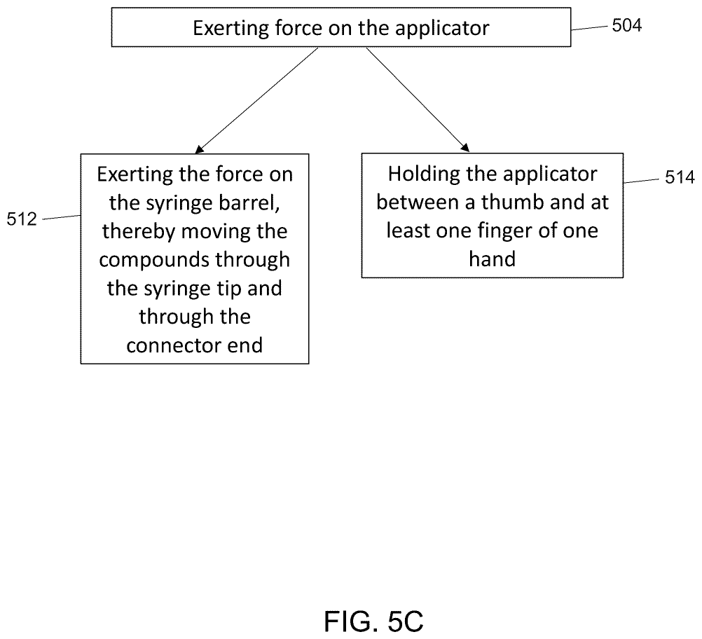

Turning now to FIG. 5, a method 500 for delivering one or more topical compounds is also disclosed herein. The method 500 comprises obtaining, at a block 502, an applicator having one or more topical compounds to be delivered, the applicator comprising a cap and a membrane, the cap comprising one end connected to the membrane, the cap comprising an inner area; exerting, at a block 504, force on the applicator to move the one or more topical compounds through the inner area and through the one end, such that at least one portion of the one or more topical compounds are extruded through the membrane; and positioning, at a block 506, the applicator adjacent to a surface of mammalian tissue such that the at least one portion of the one or more topical compounds contacts the surface, thereby delivering the one or more topical compounds.

In at least a further embodiment, the applicator comprises a syringe, which may be any syringe described above herein. The syringe comprises a barrel and a syringe tip, which may be any syringe tip described above herein. The cap may further comprise a connector end (e.g., any of the connector ends described above herein) that is configured to attach (e.g., via a releasable connection) to the syringe tip. Additionally, the aforementioned one end may be, for instance, any of the delivery ends described above herein.

Thus, as shown in FIG. 5B, the method 500 may further comprise engaging, at a block 508, the membrane with the one end of the cap; and/or engaging, at a block 510, the connector end of the cap with a syringe tip of a syringe.

Further, as shown in FIG. 5C, the aforementioned exerting step at the block 504 may further comprise exerting, at a block 512, the force on the barrel of the syringe, thereby moving the one or more topical compounds through the syringe tip and through the connector end. The exerting step may additionally comprise holding, at a block 514, the applicator between a thumb and at least one finger of one hand. A skilled artisan will appreciate that a user of the method may hold the applicator in such fashion to provide one or more benefits, as mentioned above herein, when compared with using the thumb to exert force on the syringe plunger to depress the plunger into the barrel, thereby ejecting the one or more compounds inside the barrel through a needle attached to the syringe tip.

The aforementioned one or more topical compounds in the applicator may be any of the compounds described above herein, including, for instance, triamcinolone, fluorouracil (also known as “5-FU”), tranexamic acid (also known as “TXA”), azelaic acid, one or more bleaching agents (e.g., hydroquinone), one or more topical numbing agents (e.g., lidocaine, bupivacaine), one or more topical antibiotics, one or more dyes or stains (e.g., bonnet blue, gentian violet), one or more topical anti-inflammatory compounds such as nonsteroidal anti-inflammatory drugs (e.g., diclofenac, ibuprofen, Toradol), vitamins (e.g., vitamin A, vitamin B12, vitamin E, vitamin K), one or more topical steroids, one or more topical darkening agents for conditions such as hypopigmented scars (e.g., bimatoprost), anti-oxidants (e.g., N-acetyl-cysteine, glutathione), one or more micro-dosed compounds (e.g., botulinum toxin, hyaluronic acid, other compounds used in micro-needling reservoir treatments), one or more skincare compounds, one or more cosmetic compounds, one or more nutraceutical compounds, and combinations thereof.

As stated above herein, at least one embodiment of the device for applying and/or delivering topical compounds has different shapes, sizes, and/or form factors, including shapes, sizes, and/or form factors other than the cap and/or membrane shown in FIGS. 2A-2B, FIGS. 3A-3B, and/or FIGS. 4A-4C. Non-limiting examples of such devices are described in further detail below and one or more portions thereof may be combinable. For instance, specific devices with openings and/or ports, as described below, may be combined with specific applicator ends described below (e.g., eccentric ends, beveled ends, and the like). It should be appreciated that the devices described below may also be connectable and/or connected to a syringe (e.g., via a syringe tip, such as, for instance, any of the syringe tips disclosed herein). Further, the below embodiments also apply and/or deliver any of the topical compounds described above herein.

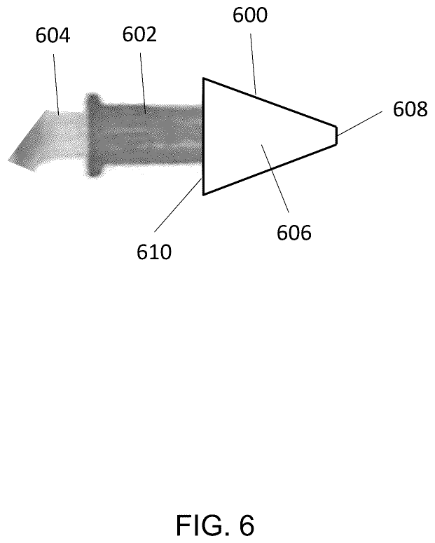

Turning now to FIG. 6, a device 600 for applying and/or delivering topical compounds is shown. The device 600 is physically connected and/or attached to a syringe tip 602 which, in turn, is physically connected and/or attached to an open connector end 604. The syringe tip 602 may be similar to, or the same as, any of the syringe tips described herein (e.g., syringe tips 106 and/or 156). The open connector end 604 may be similar to, or the same as, any of the open connector ends described herein (e.g., connector end 206). The device 600 comprises a body 606 that tapers to a tapered end 608. In other words, the tapered end is narrower than the end 610 that is attached to the open connector end 604. The tapered end 608 may have varying degrees of bluntness and/or sharpness. In at least one example, such a device 600 can be used to apply and/or deliver topical compounds, though the tapered end 608, that have a low viscosity, formulations that are more liquid in nature, and/or compounds that have a high amount of moisture and/or liquid composition.

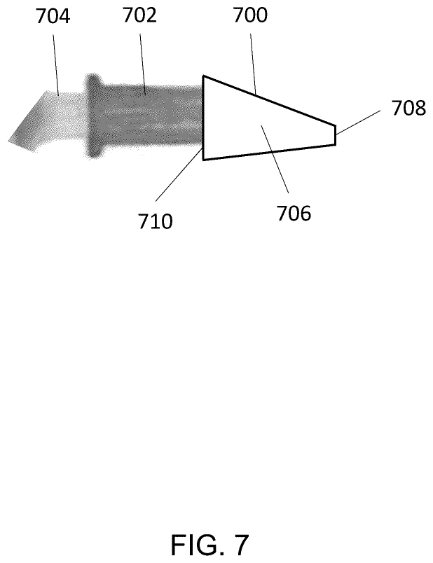

Turning now to FIG. 7, a device 700 for applying and/or delivering topical compounds is shown. The device 700 is physically connected and/or attached to a syringe tip 702 which, in turn, is physically connected and/or attached to an open connector end 704. The syringe tip 702 may be similar to, or the same as, any of the syringe tips described herein (e.g., syringe tips 106 and/or 156). The open connector end 704 may be similar to, or the same as, any of the open connector ends described herein (e.g., connector end 206). The device 700 comprises a body 706 and an eccentric or eccentric-style end 708. In other words, the eccentric end is not aligned with a center of the device 700, a center of the end 710 attached to the open connector end 704, and/or a center of the connected syringe and/or barrel (not shown). The eccentric end 708 may have varying degrees of bluntness and/or sharpness. In at least one example, such a device 700 can be used to apply and/or deliver topical compounds with a specific angle closer to the surface of human tissue (e.g., skin), resulting in a closer and/or flatter angle of application to ensure a greater amount of the topical compound is applied per administration.

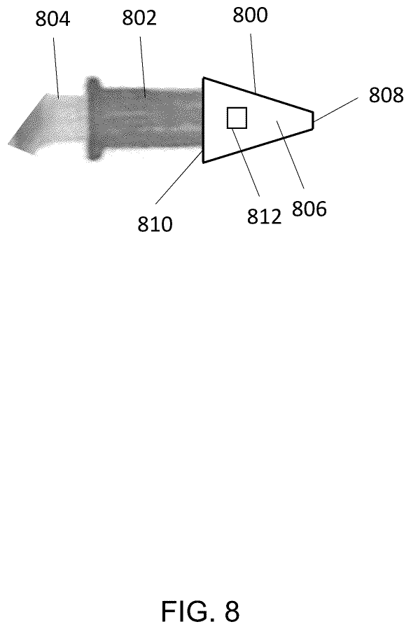

Turning now to FIG. 8, a device 800 for applying and/or delivering topical compounds is shown. The device 800 is physically connected and/or attached to a syringe tip 802 at end 810. The syringe tip 802, in turn, is physically connected and/or attached to an open connector end 804. The syringe tip 802 may be similar to, or the same as, any of the syringe tips described herein (e.g., syringe tips 106 and/or 156). The open connector end 804 may be similar to, or the same as, any of the open connector ends described herein (e.g., connector end 206). The device 800 comprises a body 806 and an end 808, each of which may be any shape, form factor, and/or configuration, including, without limitation, any of the shapes, form factors, and/or configurations described above herein. The end 808 may have varying degrees of bluntness and/or sharpness. The body 806 contains an opening and/or port 812 in a portion thereof (e.g., a side opening and/or port). Such opening and/or port 812 opens into an interior of the device 800; thus, the topical compounds to be delivered can be either accessed by the user and/or exposed to air. This opening and/or port 812 may therefore assist with reducing foaming or spraying during reconstitution of the topical compounds and/or administration thereof.

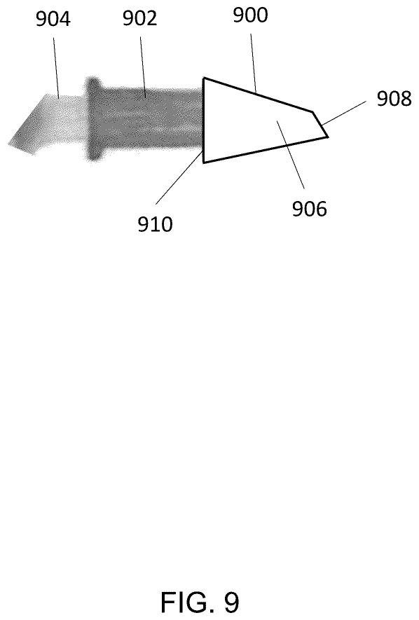

Turning now to FIG. 9, a device 900 for applying and/or delivering topical compounds is shown. The device 900 is physically connected and/or attached to a syringe tip 902 at end 910. The syringe tip 902, in turn, is physically connected and/or attached to an open connector end 904. The syringe tip 902 may be similar to, or the same as, any of the syringe tips described herein (e.g., syringe tips 106 and/or 156). The open connector end 904 may be similar to, or the same as, any of the open connector ends described herein (e.g., connector end 206). The device 900 comprises a body 906 and an angled and/or beveled end 908. The angled and/or beveled end 908 may have varying degrees of bluntness and/or sharpness. In at least one example, such a device 900 can be used to apply and/or deliver topical compounds (e.g., viscous compounds) with a specific angle of application to the surface of human tissue (e.g., skin).

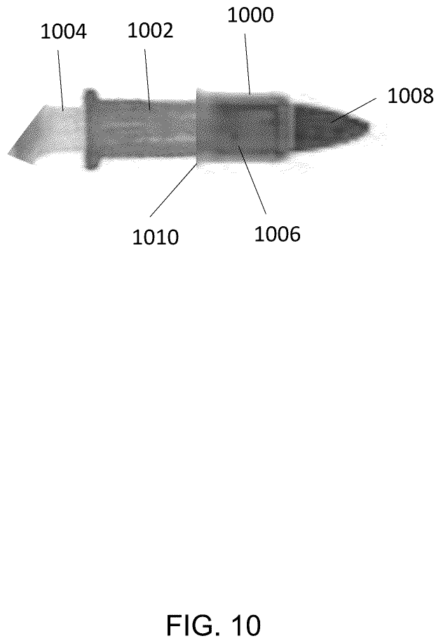

Turning now to FIG. 10, a device 1000 for applying and/or delivering topical compounds is shown. The device 1000 is physically connected and/or attached to a syringe tip 1002 at end 1010. The syringe tip 1002, in turn, is physically connected and/or attached to an open connector end 1004. The syringe tip 1002 may be similar to, or the same as, any of the syringe tips described herein (e.g., syringe tips 106 and/or 156). The open connector end 1004 may be similar to, or the same as, any of the open connector ends described herein (e.g., connector end 206). The device 1000 comprises a body 1006 and an end 1008 that narrows from the body to an elongated and/or domed point. In at least one example, the elongated and/or domed point on end 1008 is similar to a felt-tip pen in shape and/or construction (e.g., the end 1008 may be made of similar felt or felt-like material). The eccentric end 1008 may have varying degrees of bluntness. In at least one example, such a device 1000 can be used to apply and/or deliver small, controlled, and/or pre-determined amounts of topical compounds by contacting the end 1008 with the surface of human tissue (e.g., skin). The amount applied can be droplet-sized or less (e.g., brushing the topical compounds on to the surface of human tissue).

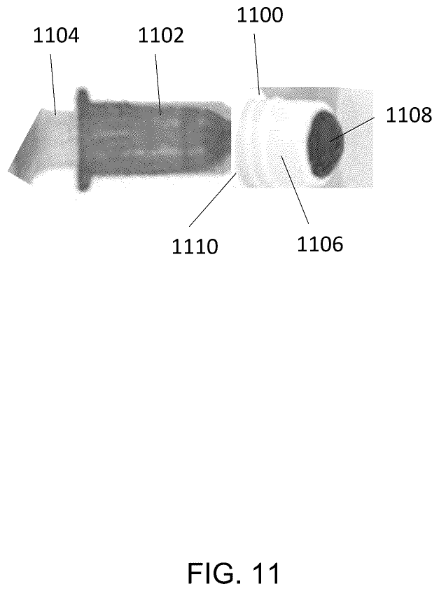

Turning now to FIG. 11, a device 1100 for applying and/or delivering topical compounds is shown. The device 1100 is physically connected and/or attached to a syringe tip 1102 at end 1110. The syringe tip 1102, in turn, is physically connected and/or attached to an open connector end 1104. The syringe tip 1102 may be similar to, or the same as, any of the syringe tips described herein (e.g., syringe tips 106 and/or 156). The open connector end 1104 may be similar to, or the same as, any of the open connector ends described herein (e.g., connector end 206). The device 1100 comprises a body 1106 and an applicator area 1108. The applicator area may be made, in whole or in part, of a sponge or sponge-like surface. The applicator area 1108 may be of any size and have varying degrees of bluntness and/or flatness. In at least one example, such a device 1100 can be used to apply and/or deliver small, controlled, and/or pre-determined amounts of topical compounds by contacting the applicator area 1108 with the surface of human tissue (e.g., skin). The amount applied can be droplet-sized or less (e.g., contacting the topical compounds on to the surface of human tissue). In at least one example, the applicator area 1108 contains amounts of the topical compounds, which can be dabbed on to the surface of human tissue. In at least another example, the device 1100 can be used with and/or deliver topical compounds having a low viscosity, formulations that are more liquid in nature, and/or compounds that have a high amount of moisture and/or liquid composition.

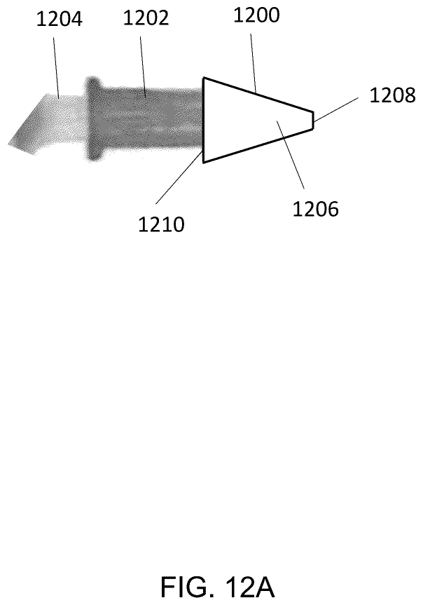

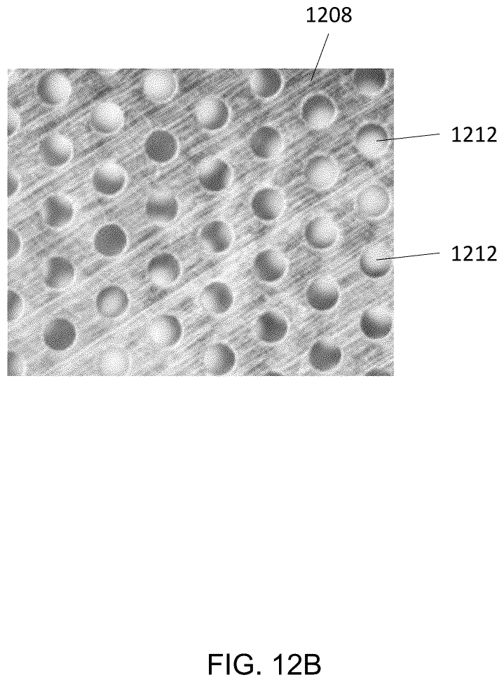

Turning now to FIG. 12A, a device 1200 for applying and/or delivering topical compounds is shown. The device 1200 is physically connected and/or attached to a syringe tip 1202 at end 1210. The syringe tip 1202, in turn, is physically connected and/or attached to an open connector end 1204. The syringe tip 1202 may be similar to, or the same as, any of the syringe tips described herein (e.g., syringe tips 106 and/or 156). The open connector end 1204 may be similar to, or the same as, any of the open connector ends described herein (e.g., connector end 206). The device 1200 comprises a body 1206 and an end 1208, each of which may be any shape, form factor, and/or configuration, including, without limitation, any of the shapes, form factors, and/or configurations described above herein. The end 1208 may have varying degrees of bluntness and/or sharpness. One or more portions of the end 1208 contains a plurality of microscopic (e.g., micron-sized and/or nanometer-sized) holes. Thus, the device 1200 can be useful for applying and/or delivering topical compounds (e.g., biological compounds and/or biological therapeutics) in micron-sized and/or nanometer-sized droplets and/or doses. FIG. 12B shows further detail of the end 1208 and the plurality of microscopic holes 1212, only two of which are labeled for convenience. The holes 1212 can be of any suitable size, including, for instance, less than 1,000 microns, less than 500 microns, less than 100 microns, less than 10 microns, less than 1 micron, less than 500 nanometers (nm), less than 100 nm, less than 10 nm, less than 1 nm, and combinations thereof.

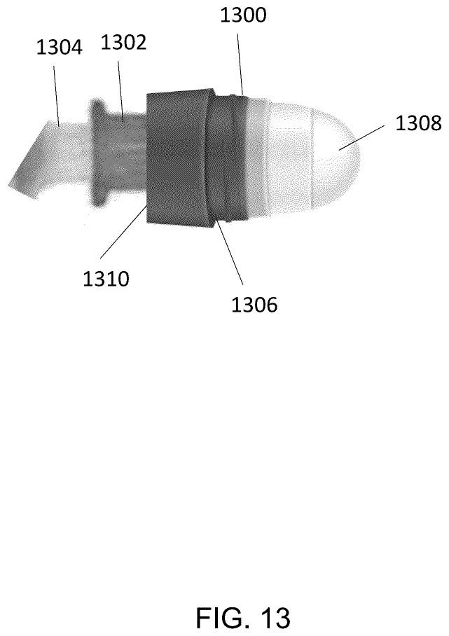

Turning now to FIG. 13, a device 1300 for applying and/or delivering topical compounds is shown. The device 1300 is physically connected and/or attached to a syringe tip 1302 at end 1310. The syringe tip 1302, in turn, is physically connected and/or attached to an open connector end 1304. The syringe tip 1302 may be similar to, or the same as, any of the syringe tips described herein (e.g., syringe tips 106 and/or 156). The open connector end 1304 may be similar to, or the same as, any of the open connector ends described herein (e.g., connector end 206). The device 1300 comprises a body 1306 and a rounded end 1308. In at least one example, the rounded end 1308 is similar to a ballpoint pen in shape and/or construction (e.g., the end 1308 may be made of similar material). The rounded end 1308 may have varying degrees of bluntness and/or flatness. In at least one example, such a device 1300 can be used to continuously apply and/or deliver small, controlled, and/or pre-determined amounts of topical compounds by contacting the rounded end 1308 with the surface of human tissue (e.g., skin). The amount applied can be droplet-sized or less (e.g., rolling the topical compounds on to the surface of human tissue).

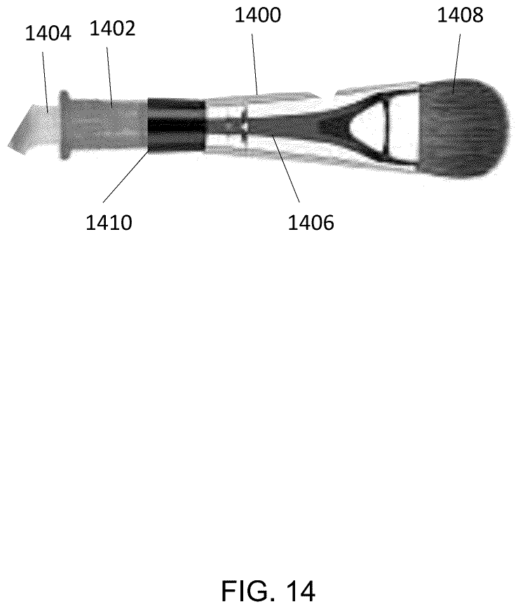

Turning now to FIG. 14, a device 1400 for applying and/or delivering topical compounds is shown. The device 1400 is physically connected and/or attached to a syringe tip 1402 at end 1410. The syringe tip 1402, in turn, is physically connected and/or attached to an open connector end 1404. The syringe tip 1402 may be similar to, or the same as, any of the syringe tips described herein (e.g., syringe tips 106 and/or 156). The open connector end 1404 may be similar to, or the same as, any of the open connector ends described herein (e.g., connector end 206). The device 1400 comprises a body 1406 and a brush end 1408. This brush end comprises a plurality of bristles, which may be made of any suitable material for applying and/or delivering the topical compounds. In at least one example, such a device 1400 can be used to apply and/or deliver topical compounds, via the brush end 1408, by brushing such compounds on a surface of human tissue (e.g., skin). Accordingly, in at least one example, the topical compounds brushed on to the surface of human tissue may comprise one or more compounds used for skincare, beauty, aesthetic, and/or makeup applications (e.g., skincare products, makeup products, fillers, dermal compounds, anti-wrinkle compounds, anti-aging compounds, and the like).

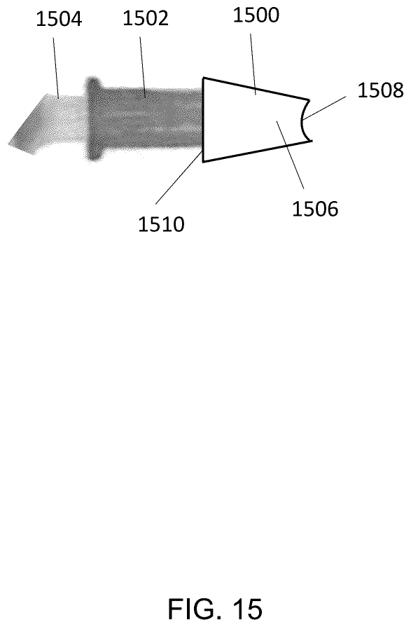

Turning now to FIG. 15, a device 1500 for applying and/or delivering topical compounds is shown. The device 1500 is physically connected and/or attached to a syringe tip 1502 at end 1510. The syringe tip 1502, in turn, is physically connected and/or attached to an open connector end 1504. The syringe tip 1502 may be similar to, or the same as, any of the syringe tips described herein (e.g., syringe tips 106 and/or 156). The open connector end 1504 may be similar to, or the same as, any of the open connector ends described herein (e.g., connector end 206). The device 1500 comprises a body 1506 and a curved end 1508 that is shaped such that it is ergonomic and/or matches to one or more rounded surfaces on human tissues (e.g., rounded portions of the fingers, arms, etc.). The curved end 1508 may therefore have a variety of curvature angles to match different sizes and/or shapes of such rounded surfaces in different users. In at least one example, such a device 1500 can be used to apply and/or deliver topical compounds directly, via curved end 1508, to the aforementioned one or more rounded surfaces, resulting in more accurate application than conventional applicators.

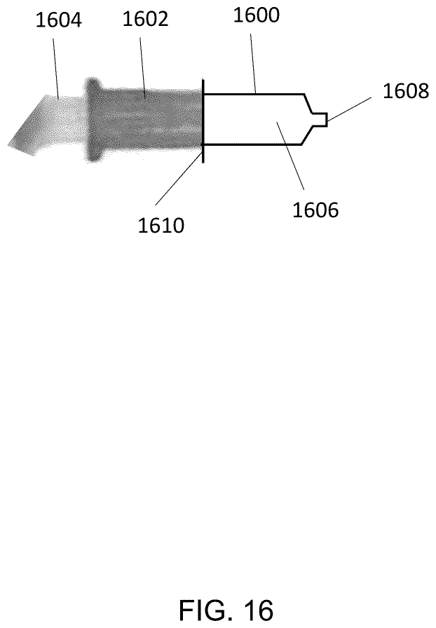

Turning now to FIG. 16, a device 1600 for applying and/or delivering topical compounds is shown. The device 1600 is physically connected and/or attached to a syringe tip 1602 at end 1610. The syringe tip 1602, in turn, is physically connected and/or attached to an open connector end 1604. The syringe tip 1602 may be similar to, or the same as, any of the syringe tips described herein (e.g., syringe tips 106 and/or 156). The open connector end 1604 may be similar to, or the same as, any of the open connector ends described herein (e.g., connector end 206). The device 1600 comprises a body 1606 and a dropper and/or dropper-style end 1608. In at least one example, the dropper and/or dropper-style end 1608 is similar to, or the same as, an eye dropper or other dropper used to administer medications in shape and/or construction. In at least one example, such a device 1600 can be used to apply and/or deliver small, controlled, and/or pre-determined amounts of topical compounds by dropping one or more droplets of the topical compounds from the dropper and/or dropper-style end 1608 on to a surface of human tissue (e.g., skin). Such controlled administration of one or more droplets can ensure more efficient and effective application of the topical compounds, reducing waste.

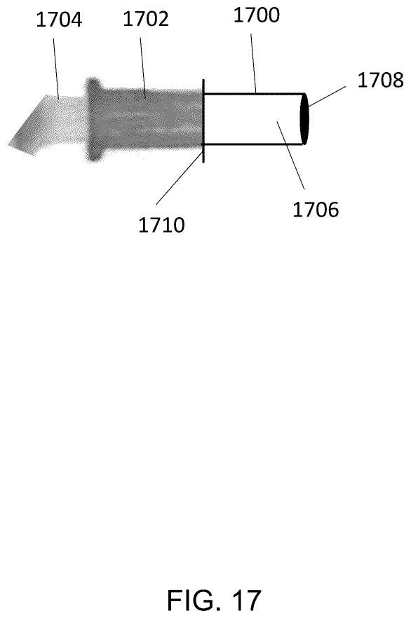

Turning now to FIG. 17, a device 1700 for applying and/or delivering topical compounds is shown. The device 1700 is physically connected and/or attached to a syringe tip 1702 at end 1710. The syringe tip 1702, in turn, is physically connected and/or attached to an open connector end 1704. The syringe tip 1702 may be similar to, or the same as, any of the syringe tips described herein (e.g., syringe tips 106 and/or 156). The open connector end 1704 may be similar to, or the same as, any of the open connector ends described herein (e.g., connector end 206). The device 1700 comprises a body 1706 and an abrasive end 1708. This abrasive end comprises one or more portions having one or more abrasive materials. Thus, in at least one example, such a device 1700 can, via the abrasive end 1708, be used for applying abrasion to a surface of human tissue (e.g., skin). Non-limiting examples of applications include removal and/or remediation of acne, scarring, and other skin irregularities (e.g., dermabrasion procedures), as well as application of topical compounds to areas of acne, scarring, and/or skin irregularities. In at least one example, the abrasive end 1708 may further comprise one or more microscopic extensions for applying the abrasion and/or applying the one or more topical compounds to surface areas to be treated. These microscopic extensions may be of any suitable size and/or length, including, for instance, less than 1,000 microns, less than 500 microns, less than 100 microns, less than 10 microns, less than 1 micron, less than 500 nanometers (nm), less than 100 nm, less than 10 nm, less than 1 nm, and combinations thereof.

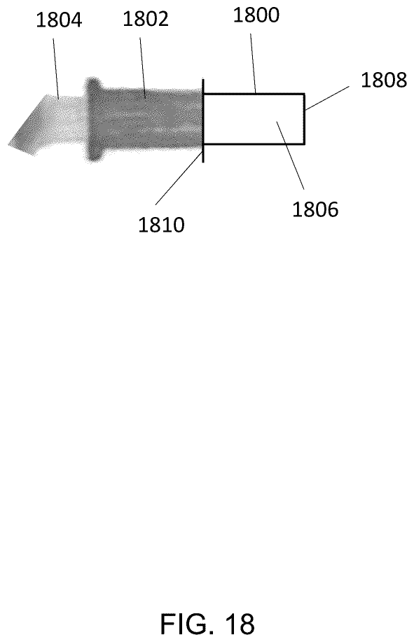

Turning now to FIG. 18, a device 1800 for applying and/or delivering topical compounds is shown. The device 1800 is physically connected and/or attached to a syringe tip 1802 at end 1810. The syringe tip 1802, in turn, is physically connected and/or attached to an open connector end 1804. The syringe tip 1802 may be similar to, or the same as, any of the syringe tips described herein (e.g., syringe tips 106 and/or 156). The open connector end 1804 may be similar to, or the same as, any of the open connector ends described herein (e.g., connector end 206). The device 1800 comprises a body 1806 and a flat end 1808 that can be placed flat against a surface of human tissue (e.g., skin). Thus, in at least one example, such a device 1800 can, via the flat end 1808, be used to apply topical compounds directly to the surface of human tissue by placing this flat end against such a surface. The flat end 1808 can also be used to apply pressure against the surface as part of the delivery and/or administration of the topical compounds. Accordingly, application via such device 1800 may result in increased and/or better absorption of the topical compounds into, and/or through, the surface of the human tissue (e.g., skin).

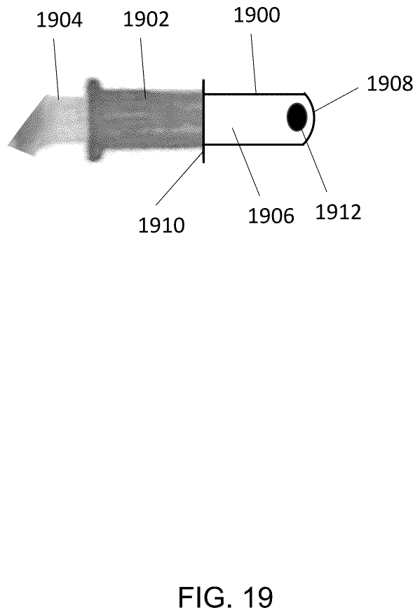

Turning now to FIG. 19, a device 1900 for applying and/or delivering topical compounds is shown. The device 1900 is physically connected and/or attached to a syringe tip 1902 at end 1910. The syringe tip 1902, in turn, is physically connected and/or attached to an open connector end 1904. The syringe tip 1902 may be similar to, or the same as, any of the syringe tips described herein (e.g., syringe tips 106 and/or 156). The open connector end 1904 may be similar to, or the same as, any of the open connector ends described herein (e.g., connector end 206). The device 1900 comprises a body 1906 and an applicator end 1908 that further comprises an internal reservoir 1912. The reservoir 1912 allows one or more topical compounds to be stored therein before administration from the applicator end 1908 to a surface of human tissue (e.g., skin). For instance, a user of the device can first move the topical compounds from the syringe and into the reservoir 1912 before topical delivery and/or application. It should be appreciated that the applicator end 1908 may therefore be of any size and have varying degrees of bluntness and/or sharpness. In at least one example, the reservoir 1912 also allows for a steady flow, via one or more fluid connections, from the reservoir through the applicator end 1908 and on to a surface of human tissue, thereby permitting smooth, steady, and consistent application of the topical compounds.

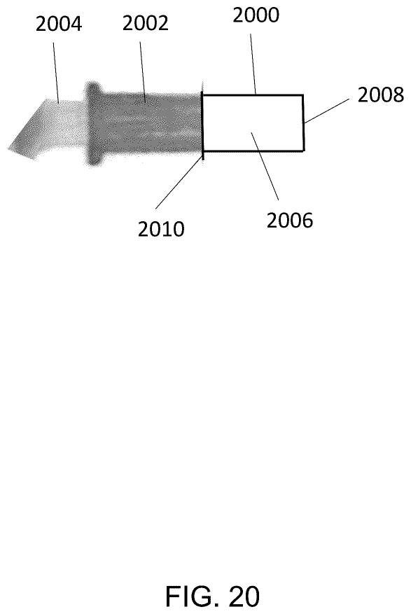

Turning now to FIG. 20, a device 2000 for applying and/or delivering topical compounds is shown. The device 2000 is physically connected and/or attached to a syringe tip 2002 at end 2010. The syringe tip 2002, in turn, is physically connected and/or attached to an open connector end 2004. The syringe tip 2002 may be similar to, or the same as, any of the syringe tips described herein (e.g., syringe tips 106 and/or 156). The open connector end 2004 may be similar to, or the same as, any of the open connector ends described herein (e.g., connector end 206). The device 2000 comprises a body 2006 and an applicator end 2008. The body 2006 and/or the applicator end 2008 have the same width as that of the syringe tip 2002 and/or the syringe and/or barrel attached to the syringe tip 2002. It should be appreciated that the applicator end 2008 may therefore be of any size, depending on the syringe tip and/or syringe used, and have varying degrees of bluntness and/or sharpness. Accordingly, in at least one example, the device 2000 can be used for applying and/or delivering topical compounds that are viscous or highly viscous in nature. Since the device 2000 has the same width as the attached syringe tip 2002 and/or syringe, the topical compounds can flow more easily from the syringe through the device and on to a surface of human tissue (e.g., skin).

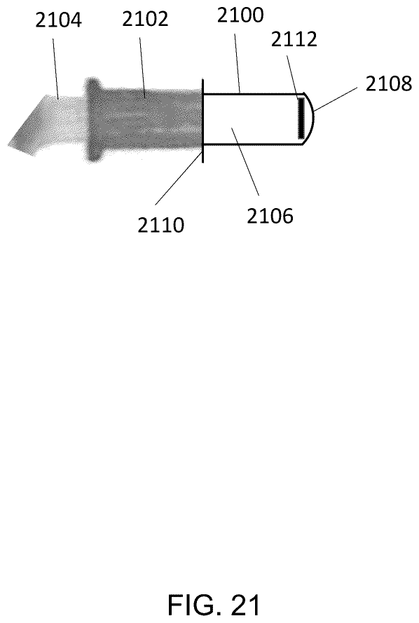

Turning now to FIG. 21, a device 2100 for applying and/or delivering topical compounds is shown. The device 2100 is physically connected and/or attached to a syringe tip 2102 at end 2110. The syringe tip 2102, in turn, is physically connected and/or attached to an open connector end 2104. The syringe tip 2102 may be similar to, or the same as, any of the syringe tips described herein (e.g., syringe tips 106 and/or 156). The open connector end 2104 may be similar to, or the same as, any of the open connector ends described herein (e.g., connector end 206). The device 2100 comprises a body 2106 and an applicator end 2108 that further comprises one or more internal filters 2112. The filter 2112 allows one or more topical compounds to be filtered (e.g., to remove unwanted particles and/or to obtain further purity) before administration from the applicator end 2108 to a surface of human tissue (e.g., skin). For instance, a user of the device can first move the topical compounds from the syringe and through the filter 2112 before topical delivery and/or application. It should be appreciated that the applicator end 2108 may therefore be of any size and have varying degrees of bluntness and/or sharpness. The filter 2112 may have any suitable pore size for medical and/or cosmetic applications, including, for instance, a 5 micron filter, a sub-5 micron filter, and combinations thereof.

Any of the devices described herein, including, without limitation, those described with reference to FIGS. 2-21, may be lubricated along one or more internal surfaces and/or cavities. Such lubrication may be achieved using any lubricant suitable for medical and/or cosmetic applications, including inert lubricants that will not interfere and/or react with the one or more topical compounds. In at least one example, the lubrication provides better flow rates of the topical compounds through the device, as well as less waste and/or better and easier application of such compounds to a surface of human tissue (e.g., skin).

Any of the devices described herein, including, without limitation, those described with reference to FIGS. 2-21, can have different lengths and/or thicknesses. Non-limiting examples of appropriate lengths include less than 1 centimeter (cm), 1-5 cm, 5-10 cm, 10-20 cm, and greater than 20 cm. Non-limiting examples of appropriate thicknesses include 0.05-0.1 millimeters (mm), 0.1-0.2 mm, 0.2-0.3 mm, 0.3-0.4 mm, 0.4-0.5 mm, and greater than 0.5 mm.

Any of the devices described herein, including, without limitation, those described with reference to FIGS. 2-21, can have one or more etchings and/or markings thereon to visualize the amount of topical compounds being stored, administered, and/or delivered. Such markings may be in milliliters (mL), cubic centimeters (cc), number of doses, or any combination thereof.

Any of the devices described herein, including, without limitation, those described with reference to FIGS. 2-21, may be made of any suitable material for medical and/or cosmetic applications, including one or more plastics, one or more polymers, and the like. Additionally, in at least one example, one or more of the devices described herein are not made of rubber and/or latex to avoid allergic reactions.

As described herein, one or more connectors and/or connection mechanisms (e.g., as used on one or more connector ends described herein) can be used with one or more embodiments of the disclosure. Non-limiting examples of such connection mechanisms include, for instance, a luer-style connector, a slip-on connection, a push-on connection, an eccentric tip, a tapered tip, a catheter tip, and combinations thereof. Other different types of connection mechanisms may also be used, non-limiting examples of which are described in further detail below.

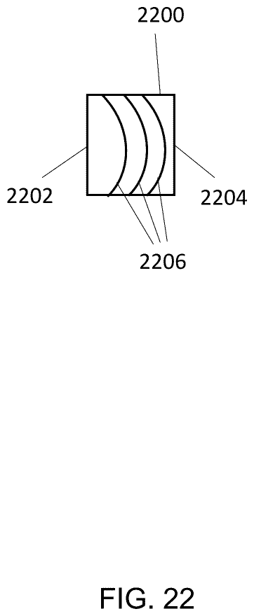

Turning now to FIG. 22, a side view of a connector 2200 is shown, with ends 2202 and 2204. Each end 2202 and 2204 may be physically attached and/or connected to a syringe or a portion thereof (e.g., a barrel), a syringe tip (e.g., any of the syringe tips described herein), any of the delivery devices or portions thereof described therein (e.g., connector end 206). Connector 2200 is compatible with non-luer-style lock connectors and comprises a plurality of threads 2206 for connection. Notably, connector 2200 does not have a hub or other protrusion. Thus, connection with connector 2200 can be accomplished via screwing the threads 2206.

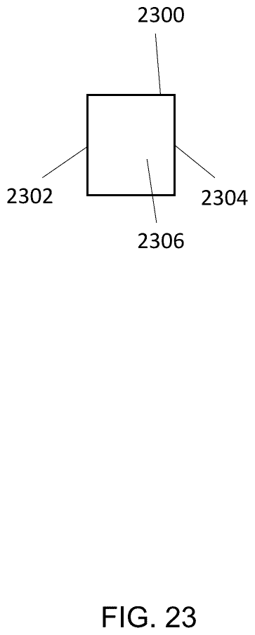

Turning now to FIG. 23, a side view of a connector 2300 is shown, with ends 2302 and 2304. Each end 2302 and 2304 may be physically attached and/or connected to a syringe or a portion thereof (e.g., a barrel), a syringe tip (e.g., any of the syringe tips described herein), any of the delivery devices or portions thereof described therein (e.g., connector end 206). Connector 2300 is compatible with non-luer-style slip connectors and comprises a surface 2306 for a slip-on connection. Notably, connector 2300 does not have threads or other aspects for a screw or twist connection. Thus, connection with connector 2300 can be accomplished via slip-on connection through contact with surface 2306.

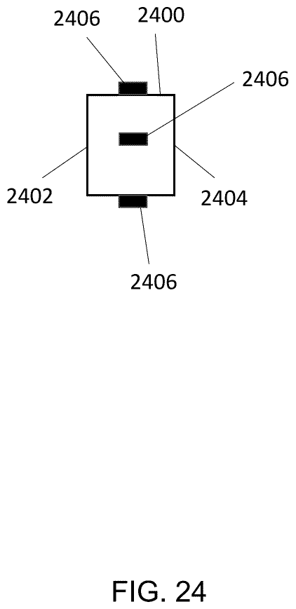

Turning now to FIG. 24, a side view of a connector 2400 is shown, with ends 2402 and 2404. Each end 2402 and 2404 may be physically attached and/or connected to a syringe or a portion thereof (e.g., a barrel), a syringe tip (e.g., any of the syringe tips described herein), any of the delivery devices or portions thereof described therein (e.g., connector end 206). Connector 2400 further comprises a plurality of tabs 2406 disposed on a surface thereof. These tabs can secure and/or hold one or more portions of the delivery device (e.g., any delivery device described herein) in place during delivery and/or administration of the topical compounds.

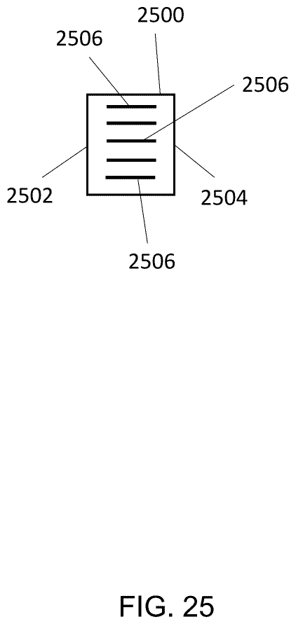

Turning now to FIG. 25, a side view of a connector 2500 is shown, with ends 2502 and 2504. Each end 2502 and 2504 may be physically attached and/or connected to a syringe or a portion thereof (e.g., a barrel), a syringe tip (e.g., any of the syringe tips described herein), any of the delivery devices or portions thereof described therein (e.g., connector end 206). Connector 2500 further comprises a plurality of ribbed aspects 2506 disposed on a surface thereof, of which only three are labeled for convenience. These ribbed aspects can secure and/or hold one or more portions of the delivery device (e.g., any delivery device described herein) in place during delivery and/or administration of the topical compounds.

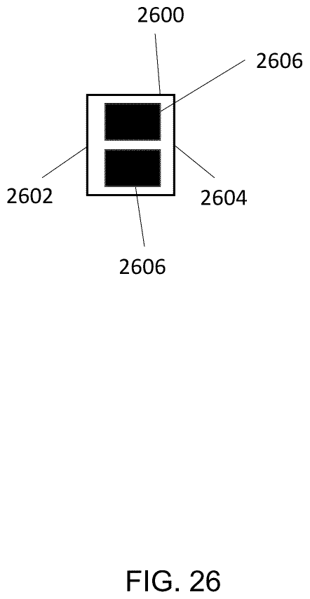

Turning now to FIG. 26, a side view of a connector 2600 is shown, with ends 2602 and 2604. Each end 2602 and 2604 may be physically attached and/or connected to a syringe or a portion thereof (e.g., a barrel), a syringe tip (e.g., any of the syringe tips described herein), any of the delivery devices or portions thereof described therein (e.g., connector end 206). Connector 2600 further comprises one or more adhesive portions 2606 disposed on a surface thereof. These adhesive portions can secure and/or hold one or more portions of the delivery device (e.g., any delivery device described herein) in place during delivery and/or administration of the topical compounds.

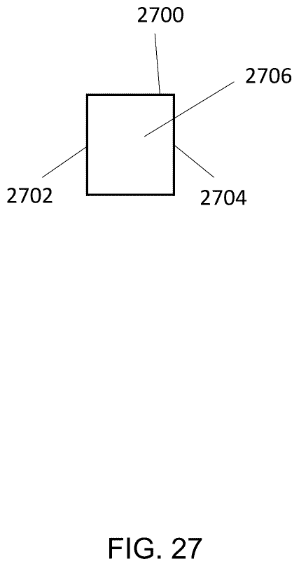

Turning now to FIG. 27, a side view of a connector 2700 is shown, with ends 2702 and 2704. Each end 2702 and 2704 may be physically attached and/or connected to a syringe or a portion thereof (e.g., a barrel), a syringe tip (e.g., any of the syringe tips described herein), any of the delivery devices or portions thereof described therein (e.g., connector end 206). Connector 2700 further comprises one or more surfaces 2706 that can secure and/or hold, via friction, one or more portions of the delivery device (e.g., any delivery device described herein) in place during delivery and/or administration of the topical compounds. In at least another example, the one or more surfaces could be inserted into one or more delivery devices or portions thereof until the shape of the delivery device or portion thereof physically prevents the one or more surfaces from being inserted further.

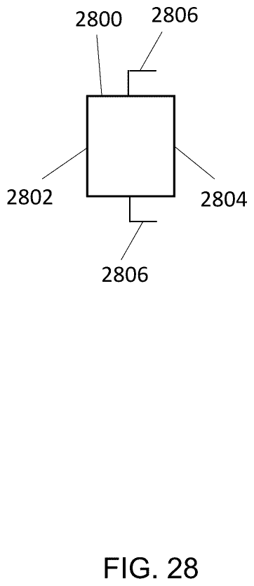

Turning now to FIG. 28, a side view of a connector 2800 is shown, with ends 2802 and 2804. Each end 2802 and 2804 may be physically attached and/or connected to a syringe or a portion thereof (e.g., a barrel), a syringe tip (e.g., any of the syringe tips described herein), any of the delivery devices or portions thereof described therein (e.g., connector end 206). Connector 2800 further comprises one or more raised sides and/or walls 2806 that can secure and/or hold one or more portions of the delivery device (e.g., any delivery device described herein) in place during delivery and/or administration of the topical compounds. In at least one example, these raised sides and/or walls can fit over one or more portions of the delivery device.

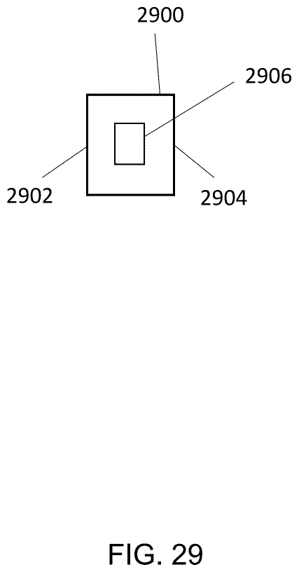

Turning now to FIG. 29, a side view of a connector 2900 is shown, with ends 2902 and 2904. Each end 2902 and 2904 may be physically attached and/or connected to a syringe or a portion thereof (e.g., a barrel), a syringe tip (e.g., any of the syringe tips described herein), any of the delivery devices or portions thereof described therein (e.g., connector end 206). Connector 2900 further comprises one or more transparent windows 2906 that can be used to visualize movement of the topical compounds through the device and/or during application and/or administration of the topical compounds.

### EXAMPLES

### Example 1: Treatment of Burns

This example describes the administration of one or more compounds and/or medications to treat one or more burn injuries.

The devices and/or applicators described herein can be used to treat one or more burn injuries on a subject. After obtaining the device and/or applicator, the barrel of the syringe can be filled with an appropriate amount and/or concentration of one or more compounds and/or medications to treat burns. Such compounds and/or medications include antibacterial agents (e.g., polysporin, which is a combination of bacitracin zinc and polymyxin B sulfate, mupirocin, neomycin, erythromycin, and the like), antiseptic agents, antifungal agents, agents containing silver (e.g., silver sulfadiazine, nanocrystalline silver, silver nitrate, and the like), agents containing bismuth, cerium nitrate, mafenide acetate, chlorhexidine (e.g., chlorhexidine gluconate), povidone-iodine (which may be in a liposomal preparation), Dakin's solution (0.025% sodium hypochlorite), gentamicin sulfate, nitrofurazone, penetration enhancing agents (e.g., glycerin, saline, sodium dodecyl sulfate, ethanol, hexane:ethanol, ethyl acetate:ethanol dimethyl sulfoxide, glycine, terpenes, and the like), one or more growth factors (e.g., PDGF, TGF-β), suitable grafts (e.g., autogenous, allografts, synthetic and/or semi-synthetic skin substitutes, and the like) and/or dressings, and combinations thereof.

After the syringe barrel has been filled, an individual (e.g., a medical professional) can apply the compound and/or medication to the burn injury site by holding the device and/or applicator between at least one finger of their hand and their thumb. The individual can then exert sufficient force on the syringe barrel to move the compound and/or medication through the syringe tip and out through a closed end of the attached membrane. Once a suitable amount of the compound and/or medication has been expelled from the syringe barrel in such a fashion, that amount can be applied to the burn injury site by positioning the device and/or applicator such that the suitable amount of the compound and/or medication contacts the burn injury site. Application of the compound and/or medication can then be repeated as necessary.

Further protection of the burn injury site, such as covering the site with a gauze or other dressing, may be done in combination with applying the compounds and/or medications described above.

Application of the compounds and/or medications described above may be performed in combination, and/or in sequence, with one or more additional therapies to treat burns and/or burn injury sites.

### Example 2: Treatment of Scars

This example describes the administration of one or more compounds and/or medications to treat one or more scars.

The devices and/or applicators described herein can be used to treat one or more scars on a subject. Such scars may result from burns, lacerations, and/or surgical incisions (e.g., due to a Cesarean section). Accordingly, scars can be painful to the subject, cause redness and/or itching, be aesthetically unappealing, and/or limit the function of tissue in and around the scarred area.

After obtaining the device and/or applicator, the barrel of the syringe can be filled with an appropriate amount and/or concentration of one or more compounds and/or medications to treat scars and/or prevent scarring. Such compounds and/or medications include steroids and/or corticosteroids (e.g., methylprednisolone, fluocinolone acetonide, triamcinolone acetonide, and the like), silicone (e.g., silicone gel), onion extract (extractum cepae), agents that act to cross-link DNA and/or prevent DNA replication (e.g., mitomycin C), agents that act as immune response modifiers (e.g., imiquimod, which can stimulate interferon, causing an increased breakdown of collagen), agents that inhibit collagen synthesis (e.g., bleomycin, which can act to inhibit collagen synthesis by decreasing stimulation by TGF-ß1), interferons (e.g., interferon alfa-2b), botulinum toxin A (BTA), suitable grafts (e.g., autogenous, allografts, synthetic and/or semi-synthetic skin substitutes, and the like) and/or dressings, and combinations thereof.

After the syringe barrel has been filled, an individual (e.g., a medical professional) can apply the compound and/or medication to the scar and/or scar area by holding the device and/or applicator between at least one finger of their hand and their thumb. The individual can then exert sufficient force on the syringe barrel to move the compound and/or medication through the syringe tip and out through a closed end of the attached membrane. Once a suitable amount of the compound and/or medication has been expelled from the syringe barrel in such a fashion, that amount can be applied to the scar and/or scar area by positioning the device and/or applicator such that the suitable amount of the compound and/or medication contacts the scar and/or scar area. Application of the compound and/or medication can then be repeated as necessary.

Application of the compounds and/or medications described above may be performed in combination, and/or in sequence, with one or more additional scar therapies, such as cryotherapy, radiotherapy (e.g., brachytherapy, X-rays, and electron beams), laser therapy (e.g., via ablative and/or non-ablative lasers), mechanical pressure therapy, microneedling procedures, dermabrasion procedures, skin peels (e.g., chemical peels), collagen replacement therapies (e.g., collagen replacement injections), surgical scar revision treatments, adhesive microporous hypoallergenic paper tape, dynamic stress-shielding devices, and combinations thereof.

Follow-up management and/or assessment of the scar and/or scarred area may be performed by using, for instance, the modified Vancouver Scar Scale (MVSS), which assesses (A) scar pigmentation on a rating scale from “0” to “2” (where a “0” rating equates to a normal pigmentation, a “1” rating equates to hypo-pigmentation, and a “2” rating equates to hyper-pigmentation), (B) vascularity on a rating scale from “0” to “5” (where a “0” rating equates to a normal color, a “1” rating equates to a pink color, a “2” rating equates to a pink to red color, a “3” rating equates to a red color, a “4” rating equates to a red to purple color, and a “5” rating equates to a purple color), (C) pliability on a rating scale from “0” to “5” (where a “0” rating equates to a normal pliability, a “1” rating equates to supple, a “2” rating equates to yielding, a “3” rating equates to firm, a “4” rating equates to banding-rope tissue, and a “5” rating equates to contracture), and (D) height on a rating scale from “0” to “3” (where a “0” rating equates to normal and/or flat, a “1” rating equates to a height of less than 2 millimeters, a “2” rating equates to a height of between 2-5 millimeters, and a “3” rating equates to a height of greater than 5 millimeters). Scar thicknesses can also be measured using, for example, linear probe ultrasounds.

### Example 3: Treatment to Improve Skin Texture, Quality, and/or Clarity

This example describes the administration of one or more compounds and/or medications to improve skin texture, quality, and/or clarity (e.g., via one or more anti-aging compounds).

After obtaining the device and/or applicator, the barrel of the syringe can be filled with an appropriate amount and/or concentration of one or more compounds and/or medications to improve skin texture, quality, and/or clarity, to treat aging skin, and/or to prevent aging of skin. Such compounds and/or medications include phenols (e.g., resveratrol), nicotinamide nucleotides (e.g., nicotinamide mononucleotides, nicotinamide adenine dinucleotides, nicotinamide ribosides), curcumin, sirolimus (which may also be called rapamycin), coenzymes (e.g., coenzyme Q10), polyamines (e.g., spermidine), catechins (e.g., epigallocatechin gallate, which may also be called epigallocatechin-3-gallate), flavonoids (e.g., quercetin, flavonols such as fisetin), stilbenoids (e.g., pterostilbene), carotenoids (e.g., crocin, astaxanthin), carnosine, anthocyanins (which may also be called anthocyans), lignans (e.g., sesamin), tyrosol, piceatannol, hesperidin, ginsenosides (e.g., ginsenoside Rg1), geroprotectants, seaweed derivatives, nanoemulsions (which may be, for instance, biphasic colloidal dispersions containing multiple immiscible liquids and one or more emulsifiers), dendrimers, polymeric nanoparticles, carbon nanotubes, phytobioactive compounds, phytoantioxidants, nano-phytoantioxidants, lycopene, antibodies (e.g., monoclonal antibodies), enzymes (e.g., catalase, superoxide dismutase, peroxidases such as glutathione peroxidase), glutathione, lipoic acid (which may also be called α-lipoic acid or thioctic acid), pimagedine (which may also be called aminoguanidine), benzo-coumarins (which may also be called dibenzo-α-pyrones) (e.g., urolithin A), trimethylglycine, hormones (e.g., growth hormone (which may also be called somatotropin or human growth hormone in its human form)), proteins (e.g., chaperone proteins such as Hsp90), steroids (e.g., pregnenolone), vitamin A (which may also be called retinol) and vitamin A forms and/or derivatives, vitamin B and vitamin B forms and/or derivatives (e.g., niacinamide or nicotinamide), vitamin C and vitamin C forms and/or derivatives, vitamin D and vitamin D forms and/or derivatives, vitamin E and vitamin E forms and/or derivatives, and combinations thereof.

After the syringe barrel has been filled, an individual (e.g., a medical professional) can apply the compound and/or medication to one or more areas of skin by holding the device and/or applicator between at least one finger of their hand and their thumb. The individual can then exert sufficient force on the syringe barrel to move the compound and/or medication through the syringe tip and out through a closed end of the attached membrane. Once a suitable amount of the compound and/or medication has been expelled from the syringe barrel in such a fashion, that amount can be applied to the one or more skin areas by positioning the device and/or applicator such that the suitable amount of the compound and/or medication contacts the one or more skin areas. Application of the compound and/or medication can then be repeated as necessary.

Application of the compounds and/or medications described above may be performed in combination, and/or in sequence, with one or more additional therapies to improve skin texture, quality, and/or clarity, to treat aging skin, and/or to prevent aging of skin.

### Example 4: Treatment Using Anti-Inflammatory Compounds

This example describes the administration of one or more compounds and/or medications to treat and/or prevent inflammation in one or more mammalian tissues (e.g., skin tissue, nerve tissue, muscle tissue, tendons, cartilage, ligaments, and the like). Accordingly, one or more of such compounds and/or medications can reduce inflammation (e.g., redness, swelling, and/or pain) in mammalian tissues. One or more of such compounds and/or medications may also block one or more substances in a mammalian subject that cause inflammation.

After obtaining the device and/or applicator, the barrel of the syringe can be filled with an appropriate amount and/or concentration of one or more compounds and/or medications to treat and/or prevent inflammation in one or more mammalian tissues and/or tissue types. Such compounds and/or medications include curcumin, colchicine, resveratrol, catechins (e.g., epigallocatechin gallate), flavonoids (e.g., quercetin, flavonols such as fisetin), anthocyanins, fatty acids (e.g., omega-3 fatty acid), phenolic compounds (e.g., oleocanthal), betaines, batalaines, capsaicin, limonin, ginsenosides (e.g., ginsenoside Rg1), luteolin, kaempferol, icariin, immune selective anti-inflammatory derivatives, selective glucocorticoid receptor agonists, resolvins, protectins, protein inhibitors (e.g., TNF inhibitors), polysaccharides (e.g., astragalus polysaccharide), minerals (e.g., selenium), enzymes (e.g., bromelain), vitamin E and vitamin E forms and/or derivatives, and combinations thereof.

After the syringe barrel has been filled, an individual (e.g., a medical professional) can apply the compound and/or medication to one or more areas of tissue that require prevention and/or treatment of inflammation by holding the device and/or applicator between at least one finger of their hand and their thumb. The individual can then exert sufficient force on the syringe barrel to move the compound and/or medication through the syringe tip and out through a closed end of the attached membrane. Once a suitable amount of the compound and/or medication has been expelled from the syringe barrel in such a fashion, that amount can be applied to the one or more areas of tissue by positioning the device and/or applicator such that the suitable amount of the compound and/or medication contacts the one or more areas of tissue. Thus, administration of the compound and/or medication can be performed on different types of tissues, such as skin tissue, neural tissue, muscle tissue, tendons, cartilage, ligaments, tissues of one or more organs, and the like. Application of the compound and/or medication can then be repeated as necessary.

Application of the compounds and/or medications described above may be performed in combination, and/or in sequence, with one or more additional therapies to treat and/or prevent inflammation.

### Example 5: Treatments Using Adhesives, Sealants, and/or Hemostatic Agents

This example describes the administration of one or more compounds and/or medications that act as adhesives, sealants, and/or hemostatic agents. Such compounds and/or medications may be used in surgeries (e.g., cartilage repair surgery, liver surgery), in wound repair treatments (e.g., to treat injuries to the spleen, eyes, and other organs), to create clots for hemostasis, and to reduce pain and/or swelling in graft procedures (e.g., skin grafts). The aforementioned compounds and/or medications can further be used in conjunction with one or more grafts, implants, and the like, including, for instance, the outside of micro-vascular anastomosis, the outside of grafts (e.g., vein grafts, bone grafts, allografts, and/or other grafts), the outside of tendons, acellular dermal matrices, the outside of reconstructions (e.g., bone reconstructions), the outside of one or more artificial and/or synthetic implants (e.g., bone substitutes, peel substitutes, and the like), the outside of one or more bioengineered interfaces and/or bioengineered devices, the outside of bioengineered interfaces and/or bioengineered devices, and combinations thereof.

After obtaining the device and/or applicator, the barrel of the syringe can be filled with an appropriate amount and/or concentration of one or more compounds and/or medications that act as adhesives, sealants, and/or hemostatic agents. Such compounds and/or medications include fibrin sealants and/or fibrin-based glues, polyurethane-based adhesives (e.g., MAR-1), polyethylene glycol polymers, albumin (e.g., bovine serum albumin), glutaraldehyde, cyanoacrylates (e.g., octyl cyanoacrylate, butyl cyanoacrylate), gelatin-based hemostatic agents, collagen (e.g., bovine collagen), cellulose (e.g., oxidized regenerated cellulose), polysaccharide spheres, plasma liquids and/or derivatives, other sealants, adhesives, and/or hemostats, and combinations thereof.

After the syringe barrel has been filled, an individual (e.g., a medical professional) can apply the compound and/or medication to one or more areas of tissue that require adhesion, sealing, and/or hemostasis by holding the device and/or applicator between at least one finger of their hand and their thumb. The individual can then exert sufficient force on the syringe barrel to move the compound and/or medication through the syringe tip and out through a closed end of the attached membrane. Once a suitable amount of the compound and/or medication has been expelled from the syringe barrel in such a fashion, that amount can be applied to the one or more areas of tissue by positioning the device and/or applicator such that the suitable amount of the compound and/or medication contacts the one or more areas of tissue. Thus, administration of the compound and/or medication can be performed on different types of tissues, such as skin tissue, neural tissue, muscle tissue, tendons, cartilage, ligaments, tissues of one or more organs, and the like. Application of the compound and/or medication can then be repeated as necessary.

Application of the compounds and/or medications described above may be performed in combination, and/or in sequence, with one or more additional therapies for promoting wound healing, treating injuries, sealing injury and/or wound sites, and/or promoting hemostasis.

### Example 6: Treatment Using Adipose-Derived Compounds and/or Tissue

This example describes the administration of one or more adipose-derived compounds and/or tissue, including nanofat, microfat, ASCs, and the like. Nanofat formulations have been described and include the following as a non-limiting example.

Briefly, nanofat formulations may be generated by first harvesting adipose tissue from a subject. For such harvesting, modified Klein solution (e.g., lidocaine 800 mg/L and adrenaline 1:1000000) is introduced into the abdomen. Adipose tissue is then harvested with a multiport 3 millimeter cannula with side holes of 1 millimeter in diameter. After harvesting, the adipose tissue is rinsed with saline, followed by filtration through a sterile cloth (e.g., a nylon cloth with 0.5 millimeter pore size) placed over a sterile container. Mechanical emulsification of the tissue is then performed by passing the tissue between two syringes connected by one or more connectors (e.g., luer-style connectors) for a minimum of 10, 20, or 30 passages. After such passages, the adipose tissue is converted into an emulsion. The emulsified tissue is then filtered over the sterile cloth again and the effluent, which is the nanofat, is collected in a sterile container.

After obtaining the device and/or applicator, the barrel of the syringe can be filled with an appropriate amount and/or concentration of one or more adipose tissue-derived compounds and/or tissue. Such compounds include nanofat, microfat, ASCs, and combinations thereof. Adipose-derived compounds and/or tissue, including nanofat, may be generally devoid of adipocytes, but contain a variety of ASCs, biological fragments (e.g., of arterioles, venules, and capillaries), growth factors (e.g., VEGF, PDGF, HGF, TGF-β, basic fibroblast growth factor (bFGF), insulin-like growth factor 1 (IGF-1), granulocyte-macrophage colony-stimulating factor (GM-CSF), and others), peptides (e.g., lipoxins, resolvins, protectins, neurotrophic factors, angiogenin, matrix metalloproteinases (“MMP” or “MMPs”) such as MMP-9, leukemia inhibitory factor, macrophage migration inhibitory factor, and the like), and/or cytokines (e.g., IL-1RA, IL-4, IL-8, IL-10, IL-11, IL-13, and the like). Such adipose-derived compounds may be liquefied and applied to one or more mammalian tissue types due to their autologous nature. In some embodiments, the size of the individual components within the adipose-derived compounds (e.g., nanofat) is between 400 to 600 μm.

After the syringe barrel has been filled, an individual (e.g., a medical professional) can apply the compound and/or tissue to one or more areas of a subject that require grafting (e.g., areas of the face, areas of the hands, the décolletage, areas of the breasts, areas of the buttocks, areas of the genitals, and the like) by holding the device and/or applicator between at least one finger of their hand and their thumb. The individual can then exert sufficient force on the syringe barrel to move the compound and/or medication through the syringe tip and out through a closed end of the attached membrane. Once a suitable amount of the compound and/or medication has been expelled from the syringe barrel in such a fashion, that amount can be applied to the one or more areas of tissue by positioning the device and/or applicator such that the suitable amount of the compound and/or medication contacts the one or more areas. Thus, administration of the compound and/or medication can be performed to different types of tissues, such as skin tissue, breast tissue, and the like. Application of the compound and/or medication can then be repeated as necessary.

Application of the compounds and/or medications described above may be performed in combination, and/or in sequence, with one or more regenerative therapies for various treatments (e.g., plastic surgery treatments including facial rejuvenation, treatments that reduce skin wrinkles, facial volume augmentation treatments, and the like).

### Example 7: Treatment Using Platelet-Rich Plasma (PRP)

This example describes the administration of one or more formulations containing platelet-rich plasma (PRP), which may also be referred to as “autologous conditioned plasma.” Such formulations may be used in orthopedics (e.g., for muscle strains, tendon conditions, muscle-fascial injuries, arthritis, and the like), dermatology (e.g., for skin rejuvenation, wound healing, alopecia, and the like), oral surgery, plastic surgery, and combinations thereof.

Generally, PRP contains concentrated platelet-rich plasma protein that is derived from whole blood. Different types of PRP exist, including (1) pure PRP (P-PRP) (which may also be referred to as leucocyte-poor PRP), which generally has no or minimal leucocytes and a low-density fibrin network, (2) leucocyte-PRP (L-PRP), which generally contains leucocytes and a low-density fibrin network, (3) pure platelet-rich fibrin (P-PRF) (which may also be referred to as leucocyte-poor PRF), which generally has no or minimal leucocytes and a high-density fibrin network, and (4) leucocyte-PRF (L-PRF), which generally contains leucocytes and a high-density fibrin network.

PRP formulations have been described and include the following two methods as non-limiting examples: (1) preparation by the PRP method, and (2) preparation by the buffy coat method.

Briefly, preparation by the PRP method begins by withdrawing and collecting whole blood. A first centrifugation is performed, after which the whole blood is separated into three layers: (1) an upper layer that contains mostly platelets and white blood cells, (2) an intermediate layer referred to as the “buffy coat,” which contains white blood cells, and (3) a bottom layer that contains mostly red blood cells. For the production of P-PRP, the upper layer and the intermediate buffy coat layer are then transferred to another tube. For the production of L-PRP, the buffy coat layer and a portion of the red blood cells are transferred to another tube. A second centrifugation is then performed to concentrate platelets. An upper portion, which generally contains platelet-poor plasma, is then removed. The pellet is then homogenized in plasma to create PRP.

Preparation by the buffy coat method begins by withdrawing and collecting whole blood. A first centrifugation is performed at relatively high speed, after which the whole blood is separated into three layers: (1) an upper layer that contains mostly platelet-poor plasma, (2) an intermediate layer referred to as the “buffy coat,” which contains platelets and white blood cells, and (3) a bottom layer that contains mostly red blood cells. The buffy coat layer is removed and subjected to a second centrifugation to separate the white blood cells. Alternatively and/or additionally, a leucocyte filtration filter may be used to filter out the leucocytes. After obtaining the device and/or applicator, the barrel of the syringe can be filled with an appropriate amount and/or concentration of one or more PRP formulations. Such formulations may include various growth factors and/or cytokines (e.g., PDGF, TGF-β, fibroblast growth factor (FGF), insulin-like growth factors, VEGF, epidermal growth factors, interleukins, keratinocyte growth factor, connective tissue growth factor, HGF, stromal cell-derived factors, endostatins, and the like).

After the syringe barrel has been filled, an individual (e.g., a medical professional) can apply the one or more PRP formulations to one or more tissues in a subject by holding the device and/or applicator between at least one finger of their hand and their thumb. The individual can then exert sufficient force on the syringe barrel to move the one or more PRP formulations through the syringe tip and out through a closed end of the attached membrane. Once a suitable amount of the one or more PRP formulations has been expelled from the syringe barrel in such a fashion, that amount can be applied to the one or more areas of tissue by positioning the device and/or applicator such that the suitable amount of the one or more PRP formulations contacts the one or more areas. Thus, administration of the one or more PRP formulations can be performed to different types of tissues, such as skin tissue, nerve tissue, muscle tissue, bone tissue, tendons, cartilage, ligaments, gum tissue, and the like. Application of the one or more PRP formulations can then be repeated as necessary.

Application of the one or more PRP formulations described above may be performed in combination, and/or in sequence, with one or more therapies and/or treatments (e.g., orthopedic treatments, dermatology treatments, oral surgery treatments, plastic surgery treatments, and the like).

### Example 8: Treatment Using Exosomes

This example describes the administration of one or more formulations containing exosomes. Such formulations may be used to treat one or more diseases or disorders (e.g., cancers, tumors, and the like).

Exemplary methods for induction of exosomes from stem cells include treatment of the stem cells with cytokines, treatment with liposome stimulation using one or more stimulant liposomes such as neutral or cationic liposomes, and/or other physical and/or biological methods previously described and referenced herein. One or more such methods may be optimized to maximize the number of exosomes present in the one or more formulations, for example, 90%, 80%, 70%, 60%, 50%, 40%, or more than 30%. Generally, methods of isolating exosomes may include one or more of differential ultracentrifugation-based techniques, size-based techniques, immunoaffinity capture-based techniques, exosome precipitation, and microfluidics-based techniques. See, e.g., Li P et al., Theranostics, 7(3): 789-804 (2017). In some embodiments, the one or more formulations include isolated exosomes and/or exogenous exosomes generated ex vivo from amniotic fluid mesenchymal stem cells (“MSC” or “MSCs”) and/or derived from MSCs of another source. In at least another embodiment, the one or more formulations include a number of engineered exosomes in a range of about 1×10² to about 1×10²⁰, for example, in a range of about 1×10² to about 1×10¹⁶, about 1×10² to about 1×10¹², about 1×10² to about 1×10¹⁰, about 1×10² to about 1×10⁶, about 1×10⁶ to about 1×10²⁰, about 1×10⁶ to about 1×10¹², about 1×10⁶ to about 1×10¹⁰, about 1×10¹⁰ to about 1×10²⁰, about 1×10¹² to about 1×10²⁰, or about 1×10¹⁶ to about 1×10²⁰.

After obtaining the device and/or applicator, the barrel of the syringe can be filled with an appropriate amount and/or concentration of one or more formulations containing exosomes. Such formulations may include one or more miRNAs (e.g., let 7a, miRNA 218, miRNA 9-5p, miRNA 19a-3p, miRNA 30a-5p, miRNA 212-5p, miRNA 323-5p, miRNA 15a, miRNA 15b, miRNA 16, miRNA 424, miRNA 497, and the like), osteoinductive factors (e.g., transforming growth factors (TGFs), bone morphogenetic proteins (BMPs), fibroblast growth factors (FGFs), insulin-like growth factors (IGFs), platelet-derived growth factors (PDGFs), osterix (OSX), one or more members of the Runx family of transcription factors, and the like), neuronal regeneration factors (e.g., c-Jun, activating transcription factor-3 (ATF-3), SRY-box containing gene 11 (Sox11), small proline-repeat protein 1A (SPRR1A), growth-associated protein-43 (GAP-43), CAP-23, and the like), immunomodulatory factors (e.g., cytokines, interferon, interleukin, antigens, growth factors, any agent that upregulates at least one member of the LXR-alpha, STAT6, and P13/Akt pathways, and the like), extracellular matrix binding factors (e.g., integrin α5, any agent that increases the binding affinity or rate to one or more components of the extracellular matrix and/or extracellular matrix-derivative peptides in a dose-dependent manner), and the like. In some embodiments, the components of the extracellular matrix include one or more of proteins (e.g., collagen, elastin, fibrin, and the like), glycoproteins (e.g., fibronectins, laminins, and the like), proteoglycans, polysaccharides (e.g., hyaluronic acid, alginate, heparin functionalized with extracellular matrix proteins or extracellular matrix-derivative peptide motifs, polylactic acid (PLA) functionalized with extracellular matrix proteins or extracellular matrix-derivative peptide motifs, polyglycolic acid (PGA) functionalized with extracellular matrix proteins or extracellular matrix-derivative peptide motifs, and the like), collagen type I (COL1) protein, and/or fibronectin 1 (FN1) protein.

The one or more formulations may also include one or more carriers, including polymer carriers (e.g., a biodegradable polymer carrier), biocompatible polymers, and/or oligomers. Non-limiting examples of biocompatible polymers or oligomers include, but are not limited to, alginate, agarose, hyaluronic acid/hyaluronan, polyethylene glycol, poly(lactic acid), poly(vinyl alcohol), polyanhydrides, poly(glycolic acid), collagen, gelatin, heparin, glycosaminoglycans, saccharides (e.g., glucose, galactose, fructose, lactose, and sucrose), self-assembling peptides, and the like. The carriers may be present in an amount of 1% to 20% by weight based on the total weight of the formulation. For instance, the carrier can be present in the amount of 1 wt % to 15 wt %, 1 wt % to 10 wt %, 1 wt % to 5 wt %, 5 wt % to 20 wt %, 5 wt % to 15 wt %, 5 wt % to 10 wt %, 10 wt % to 20 wt %, 10 wt % to 15 wt %, or 15 wt % to 20 wt %, based on the total weight of the formulation.

After the syringe barrel has been filled, an individual (e.g., a medical professional) can apply the one or more formulations containing exosomes to one or more tissues in a subject by holding the device and/or applicator between at least one finger of their hand and their thumb. The individual can then exert sufficient force on the syringe barrel to move the one or more formulations through the syringe tip and out through a closed end of the attached membrane. Once a suitable amount of the one or more formulations has been expelled from the syringe barrel in such a fashion, that amount can be applied to the one or more areas of tissue by positioning the device and/or applicator such that the suitable amount of the one or more formulations contacts the one or more areas. Thus, administration of the one or more formulations can be performed to different types of tissues, such as skin tissue, nerve tissue, muscle tissue, bone tissue, tendons, cartilage, ligaments, gum tissue, and the like. Application of the one or more formulations containing exosomes can then be repeated as necessary.

Application of the one or more formulations described above may be performed in combination, and/or in sequence, with one or more other therapies and/or treatments (e.g., cancer treatments, bone reconstruction treatments, bone regeneration treatments, skin treatments, and the like).

In summary, embodiments of the disclosure described herein enable the application and/or delivery of one or more topical compounds in a controlled and precise way, preventing over-application of the compounds and ensuring application to the desired area. Embodiments of the disclosure are configured to connect to one or more types of known syringes via one or more types of known syringe tips. Users may hold the syringe as a pen or pencil, exerting force on an outside surface of the syringe barrel to apply the one or more topical compounds.

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

I claim:

1. A device for delivering one or more topical compounds to a surface of mammalian tissue, the device comprising:
a hollow portion;
an ejector; and
a tip portion,
wherein the hollow portion is configured to hold the one or more topical compounds to be delivered to the surface of mammalian tissue,
wherein the tip portion is fluidly connected to the hollow portion, and
wherein the ejector physically moves at least one portion of the one or more topical compounds from the hollow portion through the tip portion, to deliver the one or more topical compounds to the surface of mammalian tissue.

2. The device of claim 1, wherein the tip portion is tapered such that the tip end is narrower in width than the hollow portion.

3. The device of claim 1, wherein the tip portion is eccentric with respect to the hollow portion.

4. The device of claim 1, wherein the tip portion further comprises one or more openings and/or one or more ports.

5. The device of claim 1, wherein the tip portion is angled and/or beveled.

6. The device of claim 1, wherein the tip portion narrows to an elongated and/or domed end.

7. The device of claim 1, wherein the tip portion comprises an application area, wherein the application area comprises one or more sponges.

8. The device of claim 1, wherein the tip portion comprises a plurality of microscopic holes.

9. The device of claim 8, wherein the plurality of microscopic holes comprises a plurality of micron-sized holes and/or a plurality of nanometer-sized holes.

10. The device of claim 1, wherein the tip portion comprises a rounded ball disposed at least partially inside the tip portion.

11. The device of claim 1, wherein the tip portion comprises one or more brushes.

12. The device of claim 1, wherein the tip portion comprises a curved end that is ergonomic to one or more rounded surfaces on the surface of mammalian tissue.

13. The device of claim 1, wherein the tip portion comprises an application area that dispenses the one or more topical compounds in one or more individual droplets.

14. The device of claim 1, wherein the tip portion comprises an abrasive application area for applying abrasion to the surface of mammalian tissue.

15. The device of claim 1, wherein the tip portion comprises one or more filters disposed within an interior of the tip portion.

16. The device of claim 1, wherein one or more interior surfaces of the device are pre-lubricated.

17. The device of claim 1, wherein the device comprises a plurality of markings disposed on an exterior surface of the device.

18. A device for delivering one or more topical compounds to a surface of mammalian tissue, the device comprising:
a hollow portion comprising a first connector end and an ejector; and
a tip portion comprising an applicator end and a second connector end,
wherein the hollow portion is configured to hold the one or more topical compounds to be delivered to the surface of mammalian tissue,
wherein the first connector end is fluidly connected to the second connector end,
wherein the ejector physically moves at least one portion of the one or more topical compounds through the tip portion and through the applicator end,
wherein the applicator end applies the one or more topical compounds to the surface of mammalian tissue.

19. The device of claim 18, wherein the first connector end and/or the second connector end is a non-luer-style connector.

20. The device of claim 18, wherein the first connector end and/or the second connector end is a luer-style connector.
